(19) United States
(12) Patent Application Publication
(10) Pub. No.: US 2013/0204121 A1
(43) Pub. Date: Aug. 8, 2013

(54) NANOPARTICLE-GUIDED RADIOTHERAPY

(71) Applicants: Thomas Lars Andresen, Vanlose (DK); Morten Albrechtsen, Charlottenlund (DK)

(72) Inventors: Thomas Lars Andresen, Vanlose (DK); Morten Albrechtsen, Charlottenlund (DK)

(21) Appl. No.: 13/810,588

(22) PCT Filed: Jul. 15, 2011
(86) PCT No.: PCT/EP2011/062122

§ 371 Date: Mar. 28, 2013

Related U.S. Application Data
(60) Provisional application No. 61/364,917, filed on Jul. 16, 2010.

(30) Foreign Application Priority Data
Jul. 16, 2010 (EP) 10169862.9

(51) Int. Cl. A61B 6/00; A61B 8/08; A61B 5/055; A61B 5/00; A61N 5/10; A61B 6/03

(52) U.S. Cl. CPC A61B 6/481; A61N 5/1039; A61B 6/032; A61B 5/055; A61B 6/037; A61B 5/0035; A61B 8/481; A61B 5/0075; A61B 5/0071
USPC 600/411; 600/431; 600/426

(57) ABSTRACT

The present invention relates to a method and nano-sized particles for image guided radiotherapy (IGRT) of a target tissue. More specifically, the invention relates to nano-sized particles comprising X-ray-imaging contrast agents in solid form with the ability to block x-rays, allowing for simultaneous or integrated external beam radiotherapy and imaging, e.g., using computed tomography (CT).

Each patent and non-patent reference cited in the present application is hereby incorporated by reference in its entirety.

### FIELD OF THE INVENTION

The present invention relates to a method and nano-sized particles for image guided radiotherapy (IGRT). More specifically, the invention relates to nano-sized particles comprising computed tomography (CT)-imaging contrast agents in solid form with the ability to block x-rays, which allows for simultaneous or integrated computed tomography (CT)-imaging and external beam radiotherapy.

### BACKGROUND

Cancer is a major cause of death. Presently, 1 person of 8 people dies from cancer on a worldwide basis. Another devastating fact about cancer is that it kills people of all ages. Cancer is caused by uncontrolled growth of cells, and the curative treatment of cancer aims at removing or destroying these malignant and growing cells.

### Radiotherapy

Three different methods are commonly used for treatment of cancer: Surgery, chemotherapy and radiotherapy. Radiotherapy is a commonly used method for treatment of a wide range of different cancer types and most common cancer types can be treated with radiotherapy in some way.

The major advantage of external beam radiotherapy over chemo-therapy and surgery is that it is a non-systemic and non-invasive treatment; and radiotherapy is increasingly preferred for treatment of different cancer types where surgery is difficult. Often radiotherapy is combined with the above mentioned other treatment methods for an optimal treatment of cancer.

The aim of radiotherapy is to destroy cancer tissue while saving the normal tissue. Pursuing this goal is specifically important for certain types of cancer for which radiation of normal healthy tissue leads to severe side effects. One example is in radiation therapy of prostate cancer: The prostate gland is located under the bladder and in front of the rectum, and it is vital that the external beam radiation is focused in the prostate to avoid serious side effects, such as rectal damage, incontinence and impotence. Another example is brain tumours, where the distance between the cancerous tissue and healthy tissue involved in vital functions can be very small.

Radiation treatment of tumours in tissues which move during/between treatment and imaging remains one of the major challenges in radiotherapy. The movement can for example be caused by differences in organ filling or movements while breathing. To overcome this problem, patients suffering from lung cancer are instructed not to breathe during radiation therapy. However, for a number of other types of cancers the treatment is further complicated because the tumours can be located adjacent to or inside tissues which are subject of involuntary movement.

### Imaging

In order to save normal tissue and avoid harmful side-effects of radiation in healthy tissue, it is of utmost importance to obtain a clear definition of the target volume of malignant cells compared to normal healthy cells.

The definition of malignant cells is obtained by use of different imaging modalities. Therefore, imaging is a cornerstone in radiotherapy. Today, the major imaging modalities are computed tomography (CT)-imaging, magnetic resonance imaging (MRI), positron emission tomography (PET) imaging, and single photon emission computed tomography (SPECT) imaging.

CT-imaging is a method wherein a three dimensional definition of an object is obtained from a large series of two-dimensional X-ray images taken from different angles. CT-imaging produces a volume of data which can be manipulated, in order to demonstrate various bodily structures based on their ability to block the X-ray beam. Modern scanners allow this volume of data to be reformatted in various planes and obtain a volumetric (3D) representation of structures. CT-imaging is among the most convenient imaging/diagnostic tools in hospitals today in terms of availability, efficiency and cost.

Often, the different imaging modalities are combined in order to obtain a three dimensional well-defined measure of the target volume for radiation therapy. For example CT-images are often supplemented by positron emission tomography (PET) and/or magnetic resonance (MR) imaging. The combination allow the information from two or more different imaging modalities to be correlated and interpreted in overlay images, leading to more precise information about the target volume of malignant cells and thereby accurate diagnoses.

### Planning, Tattooing and Image Guided Radiotherapy

An important part of a radiotherapy treatment is planning of the radiometric doses. The pattern of radiation delivery to the defined malignant target cells is determined using highly tailored computing applications to perform optimization and treatment simulation (treatment planning). The radiation dose is consistent with the 3-D shape of the tumour by controlling, or modulating, the radiation beam intensity. The radiation dose intensity is elevated near the gross tumour volume while radiation among the neighbouring normal tissue is decreased or avoided completely. The customized radiation dose is intended to maximize tumour dose while simultaneously protecting the surrounding normal tissue. This may result in better tumour targeting, lessened side effects, and improved treatment outcomes.

In general, at the time of planning, the intended area for treatment is manually outlined by the radiation oncologist. Once the area of treatment is determined, marks can be placed on the skin. The purpose of the ink marks is to align and position the patient daily for treatment to improve reproducibility of field placement. By aligning the markings with the radiation field (or its representation) in the radiation therapy treatment room, the correct placement of the treatment field can be identified. (Dawson & Sharpe 2006).

Over time, with improvement in technology—light fields with cross hairs, isocentric lasers—and with the shift to the practice of ‘tattooing’—a procedure where ink markings are replaced with a permanent mark by the application of ink just under the first layer of skin using a needle in documented locations—the reproducibility of the patient's setup improved.

In another strategy called “the on-line strategy” or Image-guided radiation therapy (IGRT), adjustments are made to patient and beam position during the treatment process, based on continuously updated information throughout the procedure. (Dawson & Sharpe 2006) The on-line approach requires a high-level of integration of both software and hardware. The advantage of this strategy is a reduction in both systematic and random errors, because planar or volumetric imaging techniques are employed to measure target position and correct target positional errors immediately prior to or during treatment delivery. IGRT allows more accurate control of radiation delivery to a target such as a tumour while reducing exposure to the surrounding or adjacent healthy tissue or organs.

### Markers for Imaging

The successful use of new techniques such as IGRT, and radiotherapy in general, is highly dependent on the quality of the imaging results and the facilitated use of markers for imaging. Markers for imaging are today an Achilles heel in the field of IGRT and diagnosis.

One example is the use of a marker-based IGRT program in the treatment of prostate cancer. Gold markers are implanted into the prostate to provide a surrogate position of the gland. Prior to each day's treatment, portal imaging system results are recorded. If the centre of mass has moved greater than 3 mm, then the couch is readjusted and a subsequent reference image is created (Jaffray et al. 1999). The drawbacks of such a strategy are that the markers have to be implanted by surgery, and that implantation is not easily performed for a number of cancer types.

Unfortunately, a number of other side-effects also impose serious limitations on the imaging. For example, the use of many current contrast agents comprising iodine or gadolinium for X-ray or MR imaging is affected by problems with short imaging time, a need for catheterization, occasional renal toxicity and poor contrast in large patients (Hainfeld et al. 2006).

To overcome the problem of short imaging time, WO 2006/084382 and Zheng et al. (2006) describe a formulation of the dissolved contrast agents in liposomes which provides a longer in vivo residence time. The contrast agents are formulations of dissolved iohexyl and gadoteridol for combined CT and MR imaging. However, because the contrast agents are dissolved and thus appears at relatively low concentration within the liposomes, the CT image quality when using this type of liposomes is relatively poor.

WO 2007/129311 further describes liposomes comprising formulations of dissolved iodinated contrast agents for CT imaging, wherein the wt/wt ratio of the contrast agent inside the liposome relative to the lipid mass can be as low as 20%. The method relies on contrast agents that are in solution or embedded in the lipid membrane and the CT image quality when using this type of liposomes is therefore poor.

WO 2004/017814 suggests the use of nanoparticulate contrast agents based on iodine, calcium, or a radiotracer for use in detecting inflammation in tissues.

Gold particles have recently been suggested as new X-ray contrast agents because of the high contrast compared to iodine. Hainfeld et. al. have described a study wherein gold nano-particles of 1.9 nm in diameter were used in combination with X-ray imaging detect angiogenic and hypervascularized tissue (Hainfeld et al. 2006). However, such small gold particles are associated with problems of fast clearance and low retention of the nano-particles in patients resulting in poor contrast and low image quality.

WO 2007/129791 describes gold nano-particles coated with polyethylene glycol (PEG) for use as X-ray contrast agents. The application describes the nano-particles as non-toxic and remaining in the blood vessels for a long time. There is no specific mentioning of methods for treatment in the application wherein healthy tissue is saved from radiation.

Chithrani et al. studied the intracellular uptake of gold particles contained in liposomes; however, for proposed use as radiation therapy enhancers (Chithrani et al., 2010).

There is presently a strong need in the field for improved methods and contrast agents for image guided radiotherapy.

### SUMMARY OF THE INVENTION

The present invention relates to a method and to nano-sized particles for image-guided radiotherapy. More specifically, the invention relates to nano-sized particles comprising computed tomography (CT)-imaging contrast agents in a solid form, which allows for safer treatment of target tissue by combined computed tomography (CT)-imaging and radiotherapy.

The present invention provides a method for treatment of a condition or disease associated with undesirable growth of cells in an individual, wherein said method comprises the steps of:
a) Providing nano-sized particles comprising a compound detectable by X-ray-based imaging, such as computed tomography (CT)-imaging,
b) Administering the nano-sized particles to said individual,
c) Recording X-ray-based images, such as computed tomography (CT)-images, of the undesirably growing cells thereby obtaining a definition of the target tissue giving the precise location of the undesirably growing cells and separation from normal tissue,
d) Using the definition of the target tissue obtained in c) to direct external beam radiotherapy to the undesirably growing cells and save normal tissue,
   wherein said compound is in solid form, and
   wherein image-recording and execution of radiotherapeutic treatment is integrated and performed sequentially or simultaneously.

The method according to the present invention can provide imaging results in a three or multi-dimensional coordinate data set, such as three dimensional or four dimensional, such as a four dimensional coordinate data set wherein the fourth dimension is time, said data set being used for the precise definition of the target tissue.

The nano-sized particles can be selected from, e.g., the group consisting of liposomes, polymersomes, dendrimers, water-soluble cross-linked polymers, hydrocolloids, micelles, coated metal particles, and coated particles where the core is a solid salt. Each member of this group represents a separate and specific embodiment.

Further, the detectable compound can comprise one or more isotopes selected from the group consisting of gold (Au), iodine (I), Gadolinium (Gd), bismuth (Bi), iron (Fe), Barium (Ba), Calcium (Ca), Magnesium (Mg). In one embodiment, the detectable compound is gold (Au) or Bismuth (Bi). In another embodiment, the detectable compound is gold (Au).

In one embodiment, the nano-sized particles comprise a detectable compound having a weight percent of at least 10%, such as at least 20%, such as at least 30%, such as at least 40%, such as at least 50%, such as at least 60%, such as at least 70%, such as at least 80%, such as at least 90%, such as at least 95%, such as between 90 and 100%, such as between 95% and 99%, compared to the total weight of the nano-sized particle excluding water within the particle.

The method according to the present invention may further comprise an imaging step wherein X-ray-based imaging, such as computed tomography (CT)-imaging, is combined with one or more imaging modalities from the group consisting of magnetic resonance imaging (MRI), positron emission tomography (PET) imaging, single photon emission computed tomography (SPECT) imaging, nuclear scintigraphy imaging, ultrasonic imaging, near-infrared imaging or fluorescence imaging.

The method according to the present invention can further allow for computed tomography (CT)-imaging during a period of 3 days or more days following administration, such as 3 to 30 days, such as 30 to 100 days, or such as 100 to 200 days, or such as 200 to 300 days, or such as 300 to 400 days.

In a preferred embodiment of the present invention, the method allows for computed tomography (CT)-imaging during a period of 3 to 120 days following administration.

The present invention also provides for a composition comprising nano-sized particles comprising a solid form of a compound detectable by X-ray imaging for use in image-guided radiotherapy of a target tissue in an individual, the target tissue comprising undesirably growing cells. The present invention also provides for a method for image-guided radiotherapy of a target tissue comprising undesirably growing cells, wherein the method comprises administration of such a composition.

In one embodiment of the composition or method, the image-guided radiotherapy comprises the steps of a) administering said composition to said individual; b) recording X-ray images of the target tissue to obtain a definition of the target tissue; and c) using the definition of the target tissue obtained in step b) to direct radiotherapy to the target tissue. Steps b) and c) can be performed either sequentially or simultaneously.

In one embodiment, the present invention provides methods and nano-sized particles for image-guided treatment of cancerous disease.

The nano-sized particles of any embodiment of the method or composition of the present invention can have a half-life in circulation of at least 1 hours, such as 2 to 4 hours, preferably at least 4 to 6 hours, such as at least 6 hours, such as at least 8 hours, such as at least 10 hours, such as at least 12 hours, such as at least 14 hours, such as at least 24 hours, such as at least 36 hours, such as at least 48 hours, such as at least 72 hours, such as at least 120 hours. Additionally or alternatively, the half-life can be between 1-72 hours, between 12-36 hours, between 1-24 hours, between 10-24 hours, between 5-15 hours, between 24-36 hours, between 24-72 hours, between 36-96 hours, between 48-96 hours, between 48-120 hours, between 72-120 hours, or between 72-168 hours.

Additionally or alternatively, the nano-sized particles can have a size of 10 to 150 nm, such as a number average diameter of 10 to 150 nm, such as a number average diameter of 10 to 50 nm, such as a number average diameter of 10 to 20 nm.

Exemplary nano-sized particles are selected from the group consisting of liposomes, polymersomes, dendrimers, water-soluble cross-linked polymers, hydrocolloids, micelles and coated metal particles, or are coated particles wherein the core is a solid salt.

In a particular embodiment, the nano-sized particles are liposomes. In another particular embodiment of any preceding embodiment, the nano-sized particles are solid, such as coated particles where the core comprises a metal and/or a solid salt.

The detectable compound of any preceding embodiment may be at least 10 weight percent, such as at least 20 weight percent, such as at least 30 weight percent, such as at least 50 weight percent, such as at least 60%, such as at least 70%, such as at least 80%, such as at least 90%, such as at least 95%, such as between 90% and 100%, such as between 95% and 99% weight percent of the nano-sized particle, excluding any water.

The detectable compound may further be in the form of a solid metal or a solid metal salt, and may comprise one or more isotopes selected from the group consisting of gold (Au), bismuth (Bi), iron (Fe), Barium (Ba), Calcium (Ca), and Magnesium (Mg). In one embodiment, the detectable compound is gold (Au) or bismuth (Bi). In another embodiment, the detectable compound is gold (Au).

In one embodiment, the nano-sized particles are obtainable by a method according to a method described in the Examples, e.g., according to a method of at least one of Examples I.a, I.b, I.c, I.d, I.e; II.a, II.b, II.c, II,d, II,e, II,f, II.g, II.h, II.i, and III.

In any embodiment of the composition or method of the invention, the target tissue may comprise tumour cells.

The administration of the composition in step a) can allow for the recording of X-ray images in step b) for at least 3 days after step a), such as for a period in the range of 3 to 120 days after step a), optionally wherein the nano-sized particles have a half-life in circulation of at least 8 hours, such as at least 10 hours, such as at least 12 hours, such as at least 24 hours, such as at least 36 hours, such as at least 120 hours.

Further, step b) may provide a three or multi-dimensional coordinate data set, such as three dimensional or four dimensional, such as a four dimensional coordinate data set, wherein the fourth dimension is time, said data set being used for the definition and treatment guidance of the target tissue.

Preferably, the X-ray imaging of any preceding embodiment is computed tomography (CT) imaging.

In a particular embodiment, the nano-sized particle may further comprise a radioactive or paramagnetic compound for one or more imaging modalities such as magnetic resonance imaging (MRI), positron emission tomography (PET) imaging, single photon emission computed tomography (SPECT) imaging or nuclear scintigraphy imaging. In such embodiments, the image-guided radiotherapy may further comprise an imaging step with one or more suitable imaging modalities, for example, magnetic resonance imaging (MRI), positron emission tomography (PET) imaging, single photon emission computed tomography (SPECT) imaging, nuclear scintigraphy imaging, ultrasonography imaging, ultrasonic imaging, near-infrared imaging and/or fluorescence imaging.

The present invention further provides nano-sized particle for use in image recording and/or external beam radiotherapy which comprises:
(i) a shell or surface coat comprising a lipid layer such as a lipid mono layer and/or a lipid bilayer,
(ii) a core comprising a contrast agent for X-ray-based imaging, such as computed tomography (CT)-imaging, selected from the group of gold (Au), bismuth (Bi), calcium (Ca), barium (Ba), and iron (Fe),
   wherein the contrast agent is in a solid form.

In one embodiment, the contrast agent is selected from gold (Au) and bismuth (Bi). In another embodiment, the contrast agent is gold (Au).

The present invention further provides methods for preparation of the nano-sized particles according to the invention.

It is further an object of the present invention to provide a system for use in a method according to the invention comprising an integrated computed tomography (CT)-imaging device for obtaining a definition of the target tissue, an integrated external beam radiation device and an integrated computer for processing data of said devices, wherein the system is capable of directing external beam radiotherapy based on the definition obtained by the computed tomography (CT)-imaging device.

### DESCRIPTION OF THE DRAWINGS

FIG. 1 illustrates exemplary nano-sized particle CT contrast agents. The nano-sized particle contrast agents can, for example, be in the form of structure (A) or (B). Structure (A) is constituted by an inner core (1) comprising a metal or solid salt contrast agent that is surrounded by a shell (2) that is composed of a material that gives the particle circulating properties, e.g. a polymer system such as PEG or lipids, either as a layered structure such as a monolayer or in the form of a liposome that can further be functionalized with PEG. The inner core (1) of structure (A) can furthermore be a water phase with precipitated salts or smaller nanostructures, e.g. gold nanoparticles, or a polymer matrix with nanostructures such as gold nanoparticles. Structure (B) is constituted by a matrix (3) giving the nano-sized particle circulating properties that further contain entrapped salts or metals that act as CT contrast agents. Both structure (A) and (B) can furthermore comprise agents, either non-covalently or covalently bound that are visible by other imaging modalities as described in the invention.

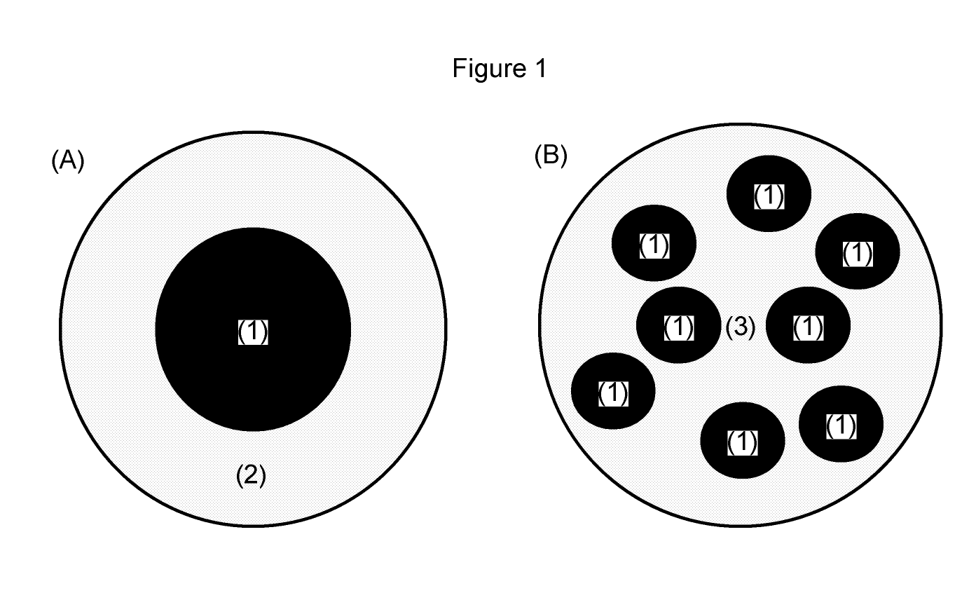

### DETAILED DESCRIPTION OF THE INVENTION

Currently, there is a need for high contrast markers facilitating the definition of the target volume of radiation prior to, or during treatment. It is an object of the present invention to provide nano-particles, methods for the use of these nano-particles and systems for integrated imaging and radiation therapy which allows for a safer, less painful and less costly imaging and radiation treatment of individuals in need thereof.

The nano-sized particles of the present invention remain in circulation long enough to locate the contrast markers to the target malignant cells. This localization of markers directly in the tissue of undesirable growth allows for precise definition of the target tissue for treatment. Further, according to the present invention, the contrast agent is detectable for a longer time period, which reduces the requirement for multiple doses and risk of toxicity.

### Nano-Sized Particles

The nano-sized particles of the present invention comprise contrast agent detectable by computed tomography (CT)-imaging.

Further, the nano-sized particles of the present invention may comprise contrast agent detectable by computed tomography (CT)-imaging and one or more additional imaging modalities.

### Contrast Agent or Detectable Compounds

The expressions “detectable compound” and “contrast agent” are used interchangeably herein. It is an object of the present invention to provide nano-sized particles comprising detectable compounds or contrast agents in solid form for X-ray and CT-imaging. Such detectable compounds are able to block or attenuate the X-ray radiation and include transition metals, rare earth metals, alkali metals, alkali earth metals, other metals, as defined by the periodic table. Such detectable compounds comprise one or more compounds selected from the group of gold (Au), gadolinium (Gd), bismuth (Bi), iron (Fe), Barium (Ba), Calcium (Ca) or Magnesium (Mg), wherein said metal or alkali metal may appear in non-oxidized or any of the existing oxidation states for the metal. These oxidation states include monovalent cations, divalent cations, trivalent cations, tetravalent cations, pentavalent cations, hexavalent cations and heptavalent cations.

In a preferred embodiment of the present invention, the detectable compound comprises one or more compounds selected from the group of gold (Au), bismuth (Bi), Gadolinium (Gd), iron (Fe), Barium (Ba) and Calcium (Ca).

In an even more preferred embodiment of the present invention, the detectable compound comprises one or more compounds selected from the group of gold (Au) and bismuth (Bi).

The contrast agent for X-ray and CT-imaging according for the present invention is comprised within the nano-sized particle, and can be non-covalently or covalently associated with the shell of the particle.

It is an object of the present invention to provide nano-sized particles comprising detectable compounds in solid form, such as a solid metal form, a solid salt form, solid alkali metal form, an aggregated, a crystallized or a precipitated form.

Preferably, the detectable compound is a solid metal form, a solid salt form or solid alkali metal form.

The amount of contrast agent comprised within the nano-sized particles according to the present invention may be quantified by the weight percent of the contrast agent relative to the total weight of the nano-sized particle, excluding any water comprised by the nano-sized particle, by defining the weight percent of the contrast agent relative to the weight of the shell of the nano-sized particle, or by quantifying the size of the contrasting agent within the prepared nano-sized particles.

In a preferred embodiment of the present invention, the detectable compound has a weight percent of at least 10% compared to the total weight of the nano-sized particle excluding water, such as at least 20%, such as at least 30%, such as at least 40%, such as at least 50%, such as at least 60%, such as at least 70%, such as at least 80% such as at least 90%, such as at least 95%, such as at least 99%, such as between 90% to 100%, such as between 95% to 99% of the weight percent relative to the total weight of the nano-sized particle excluding any water.

In another preferred embodiment of the present invention, the detectable compound has a weight percent of at least 10% compared to the total weight of the lipid comprised in the nano-sized particle, such as at least 10%, such as at least 20%, such as at least 30%, such as at least 40%, such as at least 50%, such as at least 60%, such as at least 70%, such as at least 80% such as at least 90%, such as at least 95%, such as at least 99%, such as between 90% to 100%, such as between 95% to 99% of the weight percent relative to the total weight of the lipid comprised by the nano-sized particle.

The size of the nano-sized particles or contrast agent comprised within the nano-sized particles may be measured with conventional methods of the art, such as cryo-transmission electron microscopy or dynamic light scattering.

The contrast agent comprised within the nano-sized particles of the present invention may be in a nano-scale solid form. In one embodiment, of the present invention, such nano-scale solid forms have a number average diameter in the range of 2 to 148 nm, such as 2 to 5 nm, such as 5 to 80 nm, such as 5 to 50 nm, such as 5 to 20 nm, such as 5 to 15 nm, such as 5 to 10 nm in diameter, or such as 10 to 15 nm, or such as 15 to 20 nm, or such as 20 to 30 nm, or such as 30 to 40 nm, or such as 40 to 50 nm, or such as 50 to 60 nm, or such as 60 to 70 nm, or such as 70 to 80 nm, or such as 80 to 90 nm, or such as 90 to 100 nm, or such as 100 to 110 nm, or such as 110 to 120 nm, or such as 120 to 130 nm, or such as 130 to 140 nm, or such as 140 to 150 nm.

The nano-sized particles according to the present invention may comprise one or more compounds which are detectable by several different imaging modalities. Such compounds include compounds for detection by use of computed tomography (CT)-imaging, magnetic resonance imaging (MRI), positron emission tomography (PET) imaging, single photon emission computed tomography (SPECT), nuclear scintigraphy imaging, near infrared fluorescence imaging, ultrasonography or fluorescence imaging.

In one embodiment of the present invention, the nano-sized particles further comprise one or more radioactive, paramagnetic or ferromagnetic compounds for one or more imaging modalities such as magnetic resonance imaging (MRI), positron emission tomography (PET) imaging, single photon emission computed tomography (SPECT) imaging or nuclear scintigraphy imaging. Said compounds may comprise isotopes of Copper (⁶¹Cu, ⁶⁴Cu, and ⁶⁷Cu), Indium (¹¹¹In), Technetium (⁹⁹mTc), Rhenium (¹⁸⁶Re, ¹⁸⁸Re), Gallium (⁶⁷Ga, ⁶⁸Ga), Strontium (⁸⁹Sr), Samarium (¹⁸³Sm), Ytterbium (¹⁶⁹Yb), Thallium (²⁰¹Tl), Astatine (²¹¹At), Lutetium (¹⁷⁷Lu), Actinium (²²⁵Ac) Yttrium (⁹⁰Y), Antimony (¹¹⁹Sb), Tin (¹¹⁷Sn, ¹¹³Sn), Dysprosium (¹⁸⁹Dy), Cobalt (⁸⁶Co), Iron (⁸⁹Fe), Ruthenium (⁹⁷Ru, ¹⁰³Ru), Palladium (¹⁸³Pd), Cadmium (¹¹⁸Cd), Tellurium (¹¹⁸Te, ¹²³Te) Barium (¹³¹Ba, ¹⁴⁰Ba), Gadolinium (¹⁴⁹Gd, ¹⁵¹Gd), Terbium (¹⁶⁰Tb), Gold (¹⁹⁸Au, ¹⁹⁹Au), Lanthanum (¹⁴⁰La), and Radium (²²³Ra, ²²⁴Ra) wherein said isotope of a metal radionuclide may appear in any of the existing oxidation states for the metal. These oxidation states include monovalent cations, divalent cations, trivalent cations, tetravalent cations, pentavalent cations, hexavalent cations and heptavalent cations.

Said paramagnetic or ferromagnetic compounds may also be selected from the group of Scandium (Sc), Yttrium (Y), Lanthanum (La), Titanium (Ti), Zirconium (Zr), Hafnium (Hf), Vandium (V), Niobium (Nb), Tantalum (Ta); Chromium (Cr), Molybdenium (Mo), Tungsten (W), Manganese (Mn), Technetium (Tc), Rhenium (Re), Iron (Fe), Ruthenium (Ru), Osmium (Os), Cobalt (Co), Rhodium (Rh), Iridium (Ir), Nickel (Ni), Palladium (Pd), Platinum (Pt), Copper (Cu), Silver (Ag), Gold (Au), Zinc (Zn), Cadmium (Cd), Mercury (Hg), the lanthanides such as Lathanum (La), Cerium (Ce), Praseodymium (Pr), Neodymium (Nd), Promethium (Pm), Samarium (Sm), Europium (Eu), Gadolinium (Gd), Terbium (Tb), Dysprosium (Dy), Holmium (Ho), Erbium (Er), Thulium (Tm), Ytterbium (Yb), Lutetium (Lu)) and the actinides such as Actinium (Ac), Thorium (Th), Protactinium (Pa), Uranium (U), Neptunium (Np), Plutonium (Pu), Americium (Am), Curium (Cm), Berkelium (Bk), Californium (Cf), Einsteinium (Es), Fermium (Fm), Mendelevium (Md), Nobelium (No) and Lawrencium (Lr), wherein said paramagnetic or ferromagnetic compounds may appear in any of the existing oxidation states for the metal. These oxidation states include monovalent cations, divalent cations, trivalent cations, tetravalent cations, pentavalent cations, hexavalent cations and heptavalent cations.

Said one or more radioactive, paramagnetic or ferromagnetic compounds may be covalently linked to the nano-sized particle or non-covalently associated with the nano-sized particle.

In one embodiment of the present invention, the nano-sized particles further comprise one or more fluorophore compounds for near infrared fluorescence imaging. Said compounds may comprise Alexa Fluor 680, Alexa Fluor 700, Alexa Fluor 750, Cy7, Cy5.5, IRDye 800CW, IRDye 680LT, Qdot 800 nanocrystal, Qdot 705 nanocrystal or porphyrazine compounds.

### Further Components of the Nano-Sized Particles

Nano-sized particles according to the present invention include liposomes, polymersomes, dendrimers, water-soluble cross-linked polymers, hydrogels, micelles and coated metal particles or coated solid salt.

Thus, according to the present method for treatment, the nano-sized particles can consist of a variety of components. Such nano-sized particles may or may not be known in the art. Examples of types of nano-sized particles which are useful for the method of treatment are for example gold nano-sized particles synthesized with a PEG coating or pegylated gold nanorods as described in WO2007129791 and Kim et al 2007, polymer-coated bismuth sulphide nano-sized particles as described in Rabin 2006, calcium phosphate liposome core-shell nanocomposite as described in Chu et al. 2006, dendrimers of PAMAM with entrapped gold nano-sized particles for CT imaging as described in Haba et al. 2007 and Kojima et al 2010 and other nano-sized particles comprising CT contrast agents known in the art.

The nano-sized particles of the present invention remain in circulation long enough to locate the contrast markers to the target tissue, meaning that more than 0.001% of the administered dose, in a human, reach the target tissue, such as more than 0.01%, 0.05%, 0.1%, 0.3%, 0.5%, 1%, 1.5%, 2%, 3%, 5%, or 10%. This localization of markers directly in the tissue of undesirable growth allows for precise definition of the target tissue for treatment. Further, according to the present invention, the contrast agent is detectable for a longer time period, which reduces the requirement for multiple doses and risk of toxicity.

The circulation properties of the nano-sized particle preparations can also be expressed as the half-life (T½) in humans or in animals such as rats, mice, dogs, rabbits, monkeys or pigs (preferably determined in a human), which is the amount of time necessary for one-half of the circulating nano-sized particles to be removed from plasma. This value can be calculated as a ‘true’ value (which takes into the account of distribution effect) and an ‘apparent’ elimination half-life. The half-life referred to herein is the ‘true’ value.

The half-life can be least 1 hour, such as least 2 to 4 hours, preferably at least 4 to 6 hours, such as at least 6 hours, such as at least 8 hours, such as at least 10 hours, such as at least 12 hours, such as at least 14 hours, such as at least 24 hours, such as at least 48 hours and such as at least 72 hours. Additionally or alternatively, the half-life can be between 1-72 hours, between 12-36 hours, between 1-24 hours, between 10-24 hours, between 5-15 hours, between 24-36 hours, between 24-72 hours, between 36-96 hours, between 48-96 hours, between 48-120 hours, between 72-120 hours, or between 72-168 hours.

The present invention further relates to other types of nano-sized particles for use in image recording which comprises:
(i) a shell or surface coat comprising a lipid layer such as a lipid monolayer and/or one or more lipid bilayers,
(ii) a core comprising a contrast agent for computed tomography (CT)-imaging, selected from the group of gold (Au), bismuth (Bi), calcium (Ca), barium (Ba), and iron (Fe),
   wherein said contrast agent is in a solid form and selected from the groups of detectable compounds mentioned herein.

According to the invention, liposomes, a lipid monolayer or one or more lipid bilayers can serve as shells or surface coats on the nano-sized particles according to the present invention.

Liposomes are usually characterized as nano-scaled vesicles consisting of an interior core separated from the outer environment by a membrane of one or more bilayers. The bilayer membranes or vesicles can be formed by amphiphilic molecules e.g. synthetic or natural lipids that comprise a hydrophobic and a hydrophilic domain. Bilayer membranes can also be formed by amphiphilic polymers constituting particles (e.g. polymersomes).

Liposomes can serve as carriers of an entity such as, without limitation, a chemical compound, a metal, a salt, or a radionuclide, that is capable of having a useful property or provide a useful activity. For this purpose, the liposomes are prepared to contain the desired entity in a liposome-incorporated form. The liposome incorporated entity can be associated with the exterior surface of the liposome membrane, located in the interior core of the liposome or within the bilayer of the liposome. Methods for the incorporation of metals into liposomes are e.g. surface labelling after liposome preparation, label incorporation into the lipid bilayer of preformed liposomes, surface labelling of preformed liposomes by incorporating a lipid chelator conjugate during preparation, and aqueous phase loading of preformed liposome, incorporation of a salt that forms a precipitate with the metal. The incorporation of entities into liposomes by the aqueous phase is also referred to as “encapsulating” or “entrapping” the entities.

Ideally, such liposome compositions can be prepared to include the desired entity, e.g. a chemical compound, a metal or radionuclide, (i) with a high loading efficiency, i.e., high percentage of encapsulated entity relative to the total amount of the entity used in the encapsulation process, and (ii) in a stable form, i.e., with minimal release (i.e. leakage) of the encapsulated entity upon storage or generally before the liposome reaches the site or the environment where the liposome entrapped entity is expected to apply its intended activity.

A monolayer surface coating of the nano-sized particles is ideally achieved by lipids that has high affinity interactions between the coating material and the particle surface, such as hydrophobic interactions, or through covalent conjugation, e.g. by using lipid thiols. The monolayer coating can be achieved in steps, e.g. thiol lipid conjugation followed by mololayer coating with lipids, such as phospholipids.

A bilayer surface coating or mulitiple bilayer surface coatings of the nano-sized particles is ideally achieved by high affinity interactions between the coating material and the particle surface, such as hydrophobic interactions, electrostatic interactions or due to hydrophobic effects of entropic origin.

A vesicle forming component is a synthetic or naturally-occurring amphiphatic compound which comprises a hydrophilic part and a hydrophobic part. Vesicle forming components can be used as surface-coating lipids for the purpose of the present invention, and include, for example, fatty acids, neutral fats, phosphatides, glycolipids, ceramides, sphingoglipids, aliphatic alcohols, and steroids.

Examples of suitable vesicle forming lipids or surface coating lipids useful in the present invention or the method of the present invention include, but are not limited to: phosphatidylcholines such as 1,2-dioleoyl-phosphatidylcholine, 1,2-dipalmitoyl-phosphatidylcholine, 1,2-dimyristoyl-phosphatidylcholine, 1,2-distearoyl-phosphatidylcholine, 1-oleoyl-2-palmitoyl-phosphatidylcholine, 1-oleoyl-2-stearoyl-phosphatidylcholine, 1-palmitoyl-2-oleoyl-phosphatidylcholine and 1-stearoyl-2-oleoyl-phosphatidylcholine; phosphatidylethanolamines such as 1,2-dioleoyl-phosphatidylethanolamine, 1,2-dipalmitoyl-phosphatidylethanolamine, 1,2-dimyristoyl-phosphatidylethanolamine, 1,2-distearoyl-phosphatidylethanolamine, 1-oleoyl-2-palmitoyl-phosphatidylethanolamine, 1-oleoyl-2-stearoyl-phosphatidylethanolamine, 1-palmitoyl-2-oleoyl-phosphatidylethanolamine, 1-stearoyl-2-oleoyl-phosphatidylethanolamine and N-succinyl-dioleoyl-phosphatidylethanolamine; phosphatidylserines such as 1,2-dioleoyl-phosphatidylserine, 1,2-dipalmitoyl-phosphatidylserine, 1,2-dimyristoyl-phosphatidylserine, 1,2-distearoyl-phosphatidylserine, 1-oleoyl-2-palmitoyl-phosphatidylserine, 1-oleoyl-2-stearoyl-phosphatidylserine, 1-palmitoyl-2-oleoyl-phosphatidylserine and 1-stearoyl-2-oleoyl-phosphatidylserine; phosphatidylglycerols such as 1,2-dioleoyl-phosphatidylglycerol, 1,2-dipalmitoyl-phosphatidylglycerol, 1,2-dimyristoyl-phosphatidylglycerol, 1,2-distearoyl-phosphatidylglycerol, 1-oleoyl-2-palmitoyl-phosphatidylglycerol, 1-oleoyl-2-stearoyl-phosphatidylglycerol, 1-palmitoyl-2-oleoyl-phosphatidylglycerol and 1-stearoyl-2-oleoyl-phosphatidylglycerol; pegylated lipids; pegylated phosphoholipids such as phophatidylethanolamine-N-[methoxy(polyethyleneglycol)-1000], phophatidylethanolamine-N-[methoxy(polyethyleneglycol)-2000], phophatidylethanolamine-N-[methoxy(polyethylene glycol)-3000], phophatidylethanolamine-N-[methoxy(polyethyleneglycol)-5000]; pegylated ceramides such as N-octanoyl-sphingosine-1-{succinyl[methoxy(polyethyleneglycol)1000]}, N-octanoyl-sphingosine-1-{succinyl[methoxy(polyethylene glycol)2000]}, N-octanoyl-sphingosine-1-{succinyl[methoxy(polyethyleneglycol)3000]}, N-octanoyl-sphingosine-1-{succinyl[methoxy(polyethyleneglycol)5000]}; lyso-phosphatidylcholines, lyso-phosphatidylethanolamines, lyso-phosphatidylglycerols, lyso-phosphatidylserines, ceramides; sphingolipids; glycolipids such as ganglioside GMI; glucolipids; sulphatides; phosphatidic acid, such as di-palmitoyl-glycerophosphatidic acid; palmitic fatty acids; stearic fatty acids; arachidonic fatty acids; lauric fatty acids; myristic fatty acids; lauroleic fatty acids; physeteric fatty acids; myristoleic fatty acids; palmitoleic fatty acids; petroselinic fatty acids; oleic fatty acids; isolauric fatty acids; isomyristic fatty acids; isostearic fatty acids; sterol and sterol derivatives such as cholesterol, cholesterol hemisuccinate, cholesterol sulphate, and cholesteryl-(4-trimethylammonio)-butanoate, ergosterol, lanosterol; polyoxyethylene fatty acids esters and polyoxyethylene fatty acids alcohols; polyoxyethylene fatty acids alcohol ethers; polyoxyethylated sorbitan fatty acid esters, glycerol polyethylene glycol oxy-stearate; glycerol polyethylene glycol ricinoleate; ethoxylated soybean sterols; ethoxylated castor oil; polyoxyethylene polyoxypropylene fatty acid polymers; polyoxyethylene fatty acid stearates; di-oleoyl-sn-glycerol; dipalmitoyl-succinylglycerol; 1,3-dipalmitoyl-2-succinylglycerol; 1-alkyl-2-acyl-phosphatidylcholines such as 1-hexadecyl-2-palmitoyl-phosphatidylcholine; 1-alkyl-2-acyl-phosphatidylethanolamines such as 1-hexadecyl-2-palmitoyl-phosphatidylethanolamine; 1-alkyl-2-acyl-phosphatidylserines such as 1-hexadecyl-2-palmitoyl-phosphatidylserine; 1-alkyl-2-acyl-phosphatidylglycerols such as 1-hexadecyl-2-palmitoyl-phosphatidylglycerol; 1-alkyl-2-alkyl-phosphatidylcholines such as 1-hexadecyl-2-hexadecyl-phosphatidylcholine; 1-alkyl-2-alkyl-phosphatidylethanolamines such as 1-hexadecyl-2-hexadecyl-phosphatidyl-ethanolamine; 1-alkyl-2-alkyl-phosphatidylserines such as 1-hexadecyl-2-hexadecyl-phosphatidylserine; 1-alkyl-2-alkyl-phosphatidylglycerols such as 1-hexadecyl-2-hexadecyl-phosphatidylglycerol; N-Succinyl-dioctadecylamine; palmitoylhomocysteine; lauryltrimethyl-ammonium bromide; cetyltrimethyl-ammonium bromide; myristyltrimethylammonium bromide; N-[1,2,3-dioleoyloxy)-propyl]-N,N,Ntrimethylammoniumchloride(DOTMA); 1,2-dioleoyloxy-3 (trimethyl-ammonium)propane(DOTAP); and 1,2-dioleoyl-c-(4′-trimethyl-ammonium)-butanoyl-sn-glycerol (DOTB); hecyl thiol; octyl thiol; decyl thiol; dodecyl thiol; tetradecyl thiol; hexadecyl thiol; and octadecyl thiol.

In another embodiment of the present invention, the shell of the nano-sized particle comprises amphiphatic compounds selected from the group consisting of 1,2-distearoyl-sn-glycero-3-phosphocholine (DSPC), cholesterol, and 1,2-distearoyl-sn-glycero-3-phosphoethanolamine-N-[methoxy(polyethylene glycol)-2000] (DSPE-PEG-2000) in the molar ratio of 55:40:5.

In another embodiment of the present invention, the shell of the nano-sized particle comprises amphiphatic compounds selected from the group consisting of 1,2-distearoyl-sn-glycero-3-phosphocholine (DSPC) “A”, cholesterol “B”, and 1,2-distearoyl-sn-glycero-3-phosphoethanolamine-N-[methoxy(polyethylene glycol)-2000] (DSPE-PEG-2000) “C” in the molar ratio of A:B:C, wherein A is selected from the interval 45 to 65, B is selected from the interval 35 to 45, and C is selected from the interval 2 to 12 and wherein A+B+C=100.

In one preferred embodiment of the present invention, the shell of the nano-sized particle comprises DSPC (1,2-distearoyl-sn-glycero-3-phosphocholine), CHOL (Cholesterol), DSPE-PEG-2000 (1,2-distearoyl-sn-glycero-3-phosphoethanolamine-N-[methoxy(polyethylene glycol)-2000]) in a molar ratio of 50:40:10.

In another embodiment of the present invention, the shell of the nano-sized particle comprises amphiphatic compounds selected from the group consisting of 1,2-distearoyl-sn-glycero-3-phosphocholine (DSPC) “A”, cholesterol “B”, and 1,2-distearoyl-sn-glycero-3-phosphoethanolamine-N-[methoxy(polyethylene glycol)-2000] (DSPE-PEG-2000) “C”, and 1,2-distearoyl-sn-glycero-3-phosphoethanolamine-N-[methoxy(polyethylene glycol)-2000]-TATE (DSPE-PEG-2000-RGD) “D” with the molar ratio A:B:C:D, wherein A is selected from the interval 45 to 65, B is selected from the interval 35 to 45, C is selected from the interval 5 to 13, D is selected from the interval 0 to 3, and wherein A+B+C+D=100.

In another embodiment of the present invention, the shell of the nano-sized particle comprises DSPC (1,2-distearoyl-sn-glycero-3-phosphocholine), CHOL (Cholesterol), DSPE-PEG-2000 (1,2-distearoyl-sn-glycero-3-phosphoethanolamine-N-[methoxy(polyethylene glycol)-2000]) and 1,2-distearoyl-sn-glycero-3-phosphoethanolamine-N-[methoxy(polyethylene glycol)-2000]-TATE (DSPE-PEG-2000-RGD) in a molar ratio of 50:40:9:1.

The nano-sized particles of the present invention may comprise a hydrophilic polymer such as a conjugated polyethylene glycol (PEG) component or a derivate thereof or a polysaccharide.

In one embodiment, at least one of the components of the nano-sized particle enables conjugation of proteins or other receptor affinity molecules to the vesicle forming component derivatized with the polymer.

In another embodiment, the conjugation of the polymer, such as PEG, oligosaccharides such as GM1 and GM3 or other hydrophilic polymers, to the nano-particles of the present invention composition allows for prolonged circulation time within the blood stream. Nano-sized particles comprising conjugated PEG chains on their surface are capable of extravasating leaky blood vessels.

In another embodiment of the invention, a polymer surface coating is non-covalently attached to the nano-sized particle surface through high-affinity interactions between the polymers coating and the nano-sized particle surface, such as hydrophobic interactions, electrostatic interactions or due to hydrophobic effects of entropic origin. This coating is based on a monolayer of polymers or multiple polymer layers, which can be installed using layer-by-layer techniques. Polymers can be a single polymer or block copolymers, such a diblock copolymers or triblock copolymers or mixtures hereof. One of the polymers blocks will typically be selected from polyethylene glycol (PEG), typically with a PEG molecular weight from 2000-70000 Daltons, or dextrans typically with a molecular weight between 2000 and 1000000 Daltons or hyaluronic acid typically with a molecular weight between 2000 and 1000000 Daltons. The polymers are typically combined as block copolymers in such a way that the overall polymer structure in negatively charged, allowing electrostatic interaction with a positively charged nano-sized particle surface to achieve efficient coating.

In a preferred embodiment of the present invention, the nano-sized particles comprise a conjugation of PEG, such as conjugated PEG1000, PEG2000, PEG3000, PEG 5000 or PEG10000, i.e., PEG preparations having an average molecular weight of approximately 1000, 2000, 3000, 5000 and 10000 Daltons, respectively.

### Shape and Size

The nano-sized particles according to the present invention can be quasi spherical, spherical or non-spherical such as rod-shaped.

The nano-sized particles of the present invention have a size which allows for optimized circulation and accumulation of particles in angiogenic areas, areas of undesirable cell growth or inflammatory sites. The size may according to the present invention be measured in terms of the diameter, length or width using conventional methods known in the art such as for example cryo-transmission electron microscopy or dynamic light scattering.

Thus, the nano-sized particles according to the present invention are of the size 2 to 500 nm, such as 2 to 10 nm, or such as 10 to 100 nm, such as 10 to 80 nm, such as 10 to 50 nm, such as 10 to 20 nm, such as 10 to 15 nm, or such as 15 to 20 nm, or such as 20 to 50 nm, or such as 50 to 80 nm, or such as 80 to 110 nm, or such as 110 to 140 nm, or such as 140 to 170 nm, or such as 170 to 200 nm or such as 200 to 220, or such as 220 to 250 nm, or such as 250 to 280 nm, or such as 280 to 310 nm, or such as 310 to 340 nm, or such as 340 to 370 nm, or such as 370 to 400 nm, or such as 400 to 420, or such as 420 to 450 nm, or such as 450 to 480 nm, or such as 480 to 500 nm. The size may according to the present invention be measured in terms of the diameter, length or width, including the number average diameter, length or width.

In a preferred embodiment, the nano-sized particles in the composition of the present invention have a number average diameter in the range of 10 nm to 150 nm, such as 10 to 100 nm, such as 10 to 80 nm, such as 10 to 50 nm, such as 10 nm to 30 nm, such as 10 to 20 nm, or such as 30 nm to 40 nm, or such as 40 nm to 50 nm, or such as 50 nm to 60 nm, or such as 60 nm to 70 nm, or such as 70 nm to 80 nm, or such as 90 nm to 100 nm, or such as 100 nm to 110 nm, or such as 110 nm to 120 nm, or such as 120 nm to 130 nm, or such as 130 nm to 140 nm, or such as 140 nm to 150 nm.

The contrast agent comprised in the nano-sized particles of the present invention may be in a nano-scale solid form. In one embodiment of the present invention, such nano-scale solid forms have a number average diameter of 2 to 148 nm in diameter, such as 2 to 5 nm, such as 5 to 10 nm, such as such as 5 to 80 nm, such as 5 to 50 nm, such as 5 to 20 nm, such as 5 to 15 nm, such as 10 to 15 nm, such as 15 to 20 nm, or such as 20 to 30 nm, or such as 30 to 40 nm, or such as 40 to 50 nm, or such as 50 to 60 nm, or such as 60 to 70 nm, or such as 70 to 80 nm, or such as 80 to 90 nm, or such as 90 to 100 nm, or such as 100 to 110 nm, or such as 110 to 120 nm, or such as 120 to 130 nm, or such as 130 to 140 nm, or such as 140 to 150 nm.

pH

The interior pH of the nano-sized particles according to the present invention may be controlled during synthesis of the particles or after synthesis in order to secure optimal effects. In one embodiment of the present invention or the method of the present invention, the interior pH of nano-sized particle is controlled, thus achieving a desired protonation state. Thus, according to the present invention, the interior pH of the nano-sized particle is within the range of 1 to 10, such as 1-2, for example 2-3, such as 3-4, for example 4-5, such as 5-6, for example 6-7, such as 7-8, for example 8-9, such as 9-10.

### Imaging

It is an object of the present invention to provide nano-particles and methods for imaging of the target tissue which leads to a precise definition of the target tissue.

According to the present invention, the definition of the target tissue may be described in a three or multi-dimensional coordinate data set, such as three dimensional or four dimensional, for example such as a four dimensional coordinate data set wherein the fourth dimension is time.

The methods and nano-sized particles of the present invention allow for a separation of the target tissue from healthy tissue by allowing for high quality imaging results, which lead to a more precise definition of the target tissue or cells of undesirable growth compared to healthy tissue.

Nano-sized particles according to the present invention may be used for a number of different imaging-modalities. Such imaging-modalities include computed tomography (CT)-imaging, magnetic resonance imaging (MRI), positron emission tomography (PET) imaging, single photon emission computed tomography (SPECT) imaging or nuclear scintigraphy imaging, photoacoustic imaging, ultrasonography imaging, near-infrared fluorescence imaging, fluorescence imaging or optical coherence tomography.

Preferably the nano-sized particles of the present invention are used for computed tomography (CT)-imaging.

In a more preferred embodiment, the nano-sized particles of the present invention are used for integrated, sequential or simultaneous X-ray-imaging and radiotherapy, such as integrated, sequentialor simultaneous computed tomography (CT) and radiotherapy.

In one embodiment, the X-ray imaging and radiotherapy are achieved simultaneously by use of X-ray or gamma radiation from the same radiation source. The X-ray or gamma-based radiation used for radiotherapy can thus also be used for generating X-ray images.

In another embodiment of the present invention, the nano-sized particles are for integrated, sequential or simultaneous magnetic resonance imaging (MRI) and radiotherapy, positron emission tomography (PET) imaging and radiotherapy, or single photon emission computed tomography (SPECT) and radiotherapy, and therefore comprise detectable compounds for said types of imaging as described herein.

Combination of different types of imaging modalities may also be used with the nano-sized particles of the present invention. The nano-sized particles of the present invention may be used in combinations with two imaging modalities such as computed tomography (CT)-imaging and magnetic resonance imaging (MRI), computed tomography (CT)-imaging and positron emission tomography (PET) imaging, computed tomography (CT)-imaging and single photon emission computed tomography (SPECT) imaging, computed tomography (CT)-imaging and nuclear scintigraphy imaging, computed tomography (CT)-imaging and photoacoustic imaging, computed tomography (CT)-imaging and near-infrared fluorescence imaging, computed tomography (CT)-imaging and ultrasonography imaging, computed tomography (CT)-imaging and fluorescence imaging, or such as tomography (CT)-imaging and optical coherence tomography.

The nano-sized particles of the present invention may also be used in combinations with three imaging modalities such as computed tomography (CT)-imaging, magnetic resonance imaging (MRI) and positron emission tomography (PET) imaging, or such as computed tomography (CT)-imaging, magnetic resonance imaging (MRI) and single photon emission computed tomography (SPECT) imaging, or such as computed tomography (CT)-imaging, magnetic resonance imaging (MRI) and nuclear scintigraphy imaging, or such as computed tomography (CT)-imaging, magnetic resonance imaging (MRI) and photoacoustic imaging, or such as (CT)-imaging, magnetic resonance imaging (MRI) and near-infrared fluorescence imaging, or such as computed tomography (CT)-imaging, magnetic resonance imaging (MRI) and fluorescence imaging, or such as computed tomography (CT)-imaging, magnetic resonance imaging (MRI) and ultrasonography imaging, or such as computed tomography (CT)-imaging, magnetic resonance imaging (MRI) and optical coherence tomography, or such as computed tomography (CT)-imaging positron emission tomography (PET) imaging and single photon emission computed tomography (SPECT) imaging, or such as computed tomography (CT)-imaging, positron emission tomography (PET) imaging and nuclear scintigraphy imaging, or such as computed tomography (CT)-imaging positron emission tomography (PET) imaging and photoacoustic imaging, or such as computed tomography (CT)-imaging positron emission tomography (PET) imaging and near-infrared fluorescence imaging, or such as computed tomography (CT)-imaging positron emission tomography (PET) imaging and fluorescence imaging, or such as computed tomography (CT)-imaging positron emission tomography (PET) imaging and ultrasonography imaging, computed tomography (CT)-imaging positron emission tomography (PET) imaging and optical coherence tomography, or such as computed tomography (CT)-imaging, single photon emission computed tomography (SPECT) imaging and nuclear scintigraphy imaging, or such as computed tomography (CT)-imaging, single photon emission computed tomography (SPECT) imaging and photoacoustic imaging, or such as computed tomography (CT)-imaging, single photon emission computed tomography (SPECT) imaging and near-infrared fluorescence imaging, or such as computed tomography (CT)-imaging, single photon emission computed tomography (SPECT) imaging and fluorescence imaging, computed tomography (CT)-imaging, single photon emission computed tomography (SPECT) imaging and ultrasonography imaging, or such as computed tomography (CT)-imaging, single photon emission computed tomography (SPECT) imaging and optical coherence tomography, or such as computed tomography (CT)-imaging,

The nano-sized particles of the present invention may also be used in combinations with one ore more of the above mentioned imaging modalities, such as all imaging modalities mentioned above.

It is appreciated that a planning step may be part of the methods for treatment according to the present invention. Such a planning step allows for simulation of the radiation treatment, recoding images for obtaining a clear definition of the target tissue using one or more of the above mentioned imaging modalities and adjustments of apparatus prior to radiation treatment, optimization of the 3-D shape of targeted tissue controlling, or modulating, the radiation beam's intensity. In such a planning step, the radiation dose intensity may further be optimized to be elevated near the gross tumour volume while radiation among the neighbouring normal tissue is decreased or avoided completely.

### Radiotherapeutic Treatment

The terms “radiotherapy”, “radiation therapy”, “radiotherapeutic treatment” and “radiation treatment” are used herein interchangeably and refers to therapy wherein ionizing radiation, including x-ray, gamma, proton, or ion-based radiation, is used to control or kill cells of undesirable growth. Radiotherapeutic treatment according to the present invention may be delivered by use of several techniques of radiotherapy. The radiation may be provided from a source generating a beam of radiation, such as a linear accelerator, a circular accelerator (e.g., a synchrotron or cyclotron), and/or another particle accelerator or radiation source known to those skilled in the art. Such techniques further include external beam radiation therapy in general and specific techniques of external beam radiation therapy such as conventional external beam radiotherapy (2DXRT) and stereotactic radiotherapy. Such techniques further include image guided radiotherapy (IGRT) selected from the group consisting of 3-Dimensional conformal radiotherapy (3DCRT), four-dimensional (4D) conformal radiotherapy (CRT) and intensity modulated radiotherapy (IMRT).

The needed doses of radiation, number of fractions, the shape of the radiation delivered, and frequency of the radiation therapy is according to the present invention determined by conventional methods in the art.

During current standard of radiation treatment, a safety margin is added around the target tissue to be as sure as possible to kill cancer cells while reasonably saving healthy cell. The safety margin according to current standard is typically less than 20 mm, such as about 15 mm or less, about 10 mm or less, or about 5 mm or less. The margin accounts for all uncertainties such as, but not limited to, image, movement of organ, manual incorrectness in delineation, experiences and practise. It is an objective of the invention to reduce the margin as much as possible, in order to save normal tissue while ensuring all cancer cells are killed.

It is an objective of the present invention to provide methods and nano-sized particles which allows for a more precisely defined area of target tissue, wherein the margins of healthy tissue are reduced in order to save healthy tissue. In one embodiment of the present invention, the margin can be reduced relative to current standard by at least 0.25 mm, such as at least 0.50 mm, such as at least 1 mm, such as at least 2 mm, such as at least 3 mm, such as at least 4 mm, such as at least 5 mm, such as at least 8 mm, such as at least 10 mm, such as 20 mm or more. In another embodiment, the margin is reduced to less than 20 mm, such as less than 10 mm, such as less than 8 mm, such as less than 5 mm, such as less than 4 mm, such as less than 3 mm, such as less than 2 mm, such as less than 1 mm, such as less than 0.50 mm, such as less than 0.25 mm.

According to the present invention, the image-recording and execution of radiotherapeutic treatment may be integrated, performed sequentially or simultaneously.

The methods and nano-sized particles of the present invention allows for integrated image recoding and radiation therapy, wherein the imaging is used to direct the radiation to the target tissue. According to the present invention, the location and shape of the radiation may be adjusted sequentially to imaging of the target tissue. If several imaging steps are used for defining the target tissue, the radiation beam according to the present invention may be adjusted subsequently to each imaging step in order to correct for dislocation of the target tissue. The time period between the imaging and radiation steps may be a short time-delay such as 1 microsecond to 5 seconds.

In another embodiment of the present invention, the imaging step may be done simultaneously. In another embodiment, the imaging step is done at least 1 second, such as at least 5 seconds, such as between 5 seconds to 30 days before the subsequent radiation therapy.

In some cases the target tissue needs to be defined by use of several image recordings prior to each step of radiation. In other cases, one image recording is sufficient for a definition of the target tissue which is useful for radiation. Thus according to the present invention, the sequence of imaging steps and radiation therapy may be adjusted in manner which allows for efficient treatment of target tissue while saving healthy tissue. Such sequences allow for different orders and repetition of imaging and radiation therapy.

In one embodiment of the present invention, the imaging of the target tissue may be performed simultaneously to the radiation therapy. Such simultaneous imaging and radiation therapy may be performed by utilization of the therapeutic radiation for imaging.

A more precise definition of the target tissue compared to healthy tissue allows for more intensive radiation of the target tissue and therefore fewer fractions of treatment. In one embodiment of the present invention, the radiation treatment is hypofractionated and given in large doses over fewer fractions.

The radiation therapy may be performed in several doses or fractions which may be dispersed over a time period of several days. During such treatment, the administration of nano-sized particles may be done one or more times in order to allow for imaging of the cells of undesirable growth. The radiation therapy according to the present invention may be delivered in 1 to 100 fractions, such as 1 to 5 fractions, or such as 5 to 10 fractions, or such as 10 to 20 fractions, or such as 20 to 30 fractions, or such as 30 to 40 fractions, or such as 40 to 50 fractions, or such as 50 to 60 fractions, or such as 60 to 70 fractions, or such as 70 to 80 fractions, or such as 80 to 90 fractions, or such as 90 to 100 fractions.

The one or more fractions of radiation therapy may according to the present invention further be delivered over a period of 1 to 100 days, such as 1 to 10 days, or such as 10 to 20 days, or such as 20 to 30 days, or such as 30 to 40 days, or such as 50 to 60 days, or such as 60 to 70 days, or such as 70 to 80 days, or such as 90 to 100 days.

It is further an object of the present invention to provide a system for use in a method as herein described comprising an integrated computed tomography (CT)-imaging device for obtaining a definition of the target tissue, an integrated external beam radiation device and an integrated computer for processing data of said devices, wherein the system is capable of directing external beam radiotherapy based on the definition obtained by the computed tomography (CT)-imaging device.

Diseases Associated with Undesirable Growth of Cells

The methods and nano-sized particles of the present invention relates to treatment of diseases or conditions which are associated with undesirable growth of cells.

The terms “treating”, “treatment” and “therapy” as used herein refer equally to curative therapy, prophylactic or preventative therapy and ameliorating or palliative therapy. The term includes an approach for obtaining beneficial or desired physiological results, which may be established clinically. For purposes of this invention, beneficial or desired clinical results include, but are not limited to, alleviation of symptoms, diminishment of extent of disease, stabilized (i.e., not worsening) condition, delay or slowing of progression or worsening of condition/symptoms, amelioration or palliation of the condition or symptoms, and remission (whether partial or total), whether detectable or undetectable. The term “palliation”, and variations thereof, as used herein, means that the extent and/or undesirable manifestations of a physiological condition or symptom are lessened and/or time course of the progression is slowed or lengthened, as compared to not administering compositions of the present invention.

The term “undesirable growth” includes neoplastic growth of cells in a tissue which may result in a neoplasm (i.e., a tumour), which is often characterized by increased angiogenesis. With the term “undesirable” is meant a growth of cells that may be benign, potentially malignant or malignant. A malignant cell growth may be harmful, hurtful, injurious, malevolent and/or have a lethal outcome for the individual.

Cancer is a disease characterized by undesirable growth of cells, and the present invention relates to monitoring and treatment of cancerous diseases associated with malignant neoplasia such as malignant neoplasm of lip, mouth or throat, such as malignant neoplasm of the tongue, the base of tongue, gum, floor of mouth, palate, parotid gland, major salivary glands, tonsil, oropharynx, nasopharynx, piriform sinus, hypopharynx or other parts of lip, mouth or throat or malignant neoplasms of digestive organs such as malignant neoplasms of oesophagus, stomach, small intestine, colon, rectosigmoid junction, rectum, anus and anal canal, liver and intrahepatic bile ducts, gallbladder, other parts of biliary tract, pancreas and spleen, malignant neoplasms of respiratory and intrathoracic organs such as malignant neoplasms of the nasal cavity and middle ear, accessory sinuses, larynx, trachea, bronchus and lung, thymus, heart, mediastinum and pleura, malignant neoplasms of bone and articular cartilage, such as malignant neoplasm of bone and articular cartilage of limbs, bone and articular cartilage, malignant melanoma of skin, sebaceous glands and sweat glands, malignant neoplasms of mesothelial and soft tissue such as malignant neoplasm of mesothelioma, Kaposi's sarcoma, malignant neoplasm of peripheral nerves and autonomic nervous system, malignant neoplasm of retroperitoneum and peritoneum, malignant neoplasm of connective and soft tissue such as blood vessels, bursa, cartilage, fascia, fat, ligament, lymphatic vessel, muscle, synovia, tendon, head, face and neck, abdomen, pelvis or overlapping lesions of connective and soft tissue, malignant neoplasm of breast or female genital organs such as malignant neoplasms of vulva, vagina, cervix uteri, corpus uteri, uterus, ovary, Fallopian tube, placenta or malignant neoplasms of male genital organs such as malignant neoplasms of penis, prostate, testis, malignant neoplasms of the urinary tract, such as malignant neoplasms of kidney, renal pelvis, ureter, bladder, urethra or other urinary organs, malignant neoplasms of eye, brain and other parts of central nervous system such as malignant neoplasm of eye and adnexa, meninges, brain, spinal cord, cranial nerves and other parts of central nervous system, malignant neoplasms of thyroid and other endocrine glands such as malignant neoplasm of the thyroid gland, adrenal gland, parathyroid gland, pituitary gland, craniopharyngeal duct, pineal gland, carotid body, aortic body and other paraganglia, malignant neoplasms of head, face and neck, thorax, abdomen and pelvis, secondary malignant neoplasm of lymph nodes, respiratory and digestive organs, kidney and renal pelvis, bladder and other and urinary organs, secondary malignant neoplasms of skin, brain, cerebral meninges, or other parts of nervous system, bone and bone marrow, ovary, adrenal gland, malignant neoplasms of lymphoid, haematopoietic and related tissue such as Hodgkin's disease, follicular non-Hodgkin's lymphoma, diffuse non-Hodgkin's lymphoma, peripheral and cutaneous T-cell lymphomas, non-Hodgkin's lymphoma, lymphosarcoma, malignant immunoproliferative diseases such as Waldenstrom's macroglobulinaemia, alpha heavy chain disease, gamma heavy chain disease, immunoproliferative small intestinal disease, multiple myeloma and malignant plasma cell neoplasms such as plasma cell leukaemia, plasmacytoma, solitary myeloma, lymphoid leukaemia such as acute lymphoblastic leukaemia, myeloid leukaemia, monocytic leukaemia, blast cell leukaemia, stem cell leukaemia, and other and unspecified malignant neoplasms of lymphoid, haematopoietic and related tissue such as Letterer-Siwe disease, malignant histiocytosis, malignant mast cell tumour, true histiocytic lymphoma or other types of malignant neoplasia.

Carcinoma in situ are also considered as a disease associated with undesirable cell growth. According to the present invention, a disease associated with undesirable cell growth may be carcinoma in situ of oral cavity, oesophagus, stomach, digestive organs, middle ear and respiratory system, melanoma in situ, carcinoma in situ of skin, carcinoma in situ of breast, carcinoma in situ of female or male genitals, carcinoma in situ of bladder, urinary organs or eye, thyroid and other endocrine glands, or other types of carcinoma in situ.

In a preferred embodiment, the present invention relates to undesirable growth of cells associated with lung cancer, prostate cancer, cervix or ovarian cancer.

In a more preferred embodiment, the present invention relates to undesirable growth of cells associated lung cancer or prostate cancer.

Other types of conditions or diseases associated with undesirable cell growth include extra uterine (ectopic) pregnancy, benign tumours in brain, such as benign tumours located closely to the optical nerve, glandule with overproduction of hormone, such as for example hypothalamus, bone and cartilage in relation with nerve compression, blood cells which may be killed prior to transplantation, conditions associated with large tonsils such as acute tonsillitis or adenoiditis, obstructive sleep apnoea, nasal airway obstruction, snoring, or peritonsillar abscess or hyperplasic or angiogenic eye disorders.

### Individual

Individuals according to the present invention are animal individuals. Mammal individuals, such as human individuals are regarded as part of animal individuals.

Pregnant female individuals are also regarded as individuals according to the present invention.

### Circulation

According to the present invention, the nano-sized particles may be administered in a manner allowing for circulation in the blood, lymph or cerebrospinal fluid. Such circulation of said nano-sized particles may allow for imaging of vasculature or lymph system.

The detectable compounds according to the present invention are comprised in a nano-sized particle which allows for increased circulation time, because of the protected location of the entity inside the nano-sized particle. Such protection decreases destruction and rapid excretion in vivo. By increasing the circulation time, it is ensured that the compounds comprised within the nano-sized particles reach the target tissue. A detectable compound entrapped within a long-circulating nano-sized particle can be delivered by passive targeting to a diseased site within a subject to facilitate a diagnosis thereof.

Nano-sized particles of the present invention may comprise compounds attached to the outer surface, which allows for prolonged circulation time in the blood stream. Prolonged circulation time may be obtained by decreasing the attack of the immune system soon after administration, thereby postponing clearance and preventing rupture of the nano-sized particles. Such compounds attached on the outer surface of the nano-particles include PEG, oligosaccharides such as GM1 and GM3, and hydrophilic polymers.

In a preferred embodiment of the present invention, the nano-sized particles have a shell or surface coat comprising PEG and/or a lipid layer such as a lipid mono layer and/or one or more lipid bilayers.

In another preferred embodiment of the present invention, the nano-sized particles have a shell or surface coat comprising PEG or a block co-polymer where one block is PEG and the other secures stable attachment/adhesion to the particle core. In this embodiment, the PEG molecule may, for example, may have a molecular weight between 2-70 kD.

The nano-sized particles may have a half life in circulation of at least 1 hours, such as 2 to 4 hours, preferably at least 4 to 6 hours, such as at least 6 hours, such as at least 8 hours, such as at least 10 hours, such as at least 12 hours, such as at least 14 hours, such as at least 24 hours, such as at least 36 hours, such as at least 48 hours, such as at least 72 hours, such as at least 120 hours.

### Retention in the Target Tissue

It is an objective of the present invention to provide nano-sized particles which are able to accumulate by passive targeting delivery in tissues characterized by undesirable cell growth. Such accumulation is allowed for because of the long-circulation time of the nano-sized particles and optimal size for accumulation in leaky vasculature and/or areas of non-effective lymphatic drainage system.

Exemplary target tissues include cancerous tissue such as tumours; normal tissues such as, e.g., lymph nodes, which may comprise cancer cells; foetal tissue, such as e.g., in an ectopic pregnancy; and inflammatory tissues. In one embodiment, the target tissue is cancer-related, such as a tumour.

The retention of the nano-sized particles of the present invention directly in the target tissue allows for more precise imaging of the target tissue. Since the target tissue may move during treatment, the retention of the nano-sized particles directly within the target tissue allows for continuous imaging of the precise location of the target tissue. This, in turn leads to a better definition of the areas to be treated and the saving of more healthy tissue from radiation.

It is further an object of the present invention to provide nano-sized particles which allow a long period of imaging of the target tissue after administration of the particles. Thus, according to the present invention the administration of the nano-sized particles to an individual allows for computed tomography (CT)-imaging of the target tissue during a period of 3 or more days following administration, such as 3 to 300 days or more days following administration, such as 3 to 100 days, or such as 100 to 200 days, or such as 200 to 300 days, or such as 300 to 400 days, or such as 3 to 200 days or such as 3 to 300 days or such as 3 to 400 days.

A preferred embodiment of the present invention allows for computed tomography (CT)-imaging of the target tissue during a period of 3 to 120 days following administration of the nano-sized particles.

Active- or ligand targeting delivery systems refer to nano-sized particle compositions with ligands attached on the surface targeted to cell surface antigens or receptors. Combining the properties of targeted and long-circulating liposomes in one preparation comprising a contrast compound would significantly enhance the specificity and intensity of the localization of the contrast compound in the target site e.g. a tumour.

Targeting moieties comprised in nano-sized particles allow for a higher degree of delivery and retention of the nano-sized particles in the target tissue or into target cells. This in turn leads to enhanced specificity and intensity of the detectable compound localization in the target site e.g. a tumour. Thus, the nano-sized particles provided by the present invention may further comprise targeting moieties such as saccharides, oligosaccharides, vitamins, peptides, proteins, antibodies and affibodies and other receptor binding ligands, which have specific affinity for inflammatory tissues or tissues comprising cells of undesirable growth.

An “antibody” in accordance with the present specification is defined as a protein that binds specifically to an epitope of an antigen. Such antibodies useful in the present invention may be monospecific, bispecific, trispecific, or of greater multi-specificity. For example, multi-specific antibodies may be specific for different epitopes of a cytokine, cell, or enzyme which may be present in an increased amount at the target site compared to the normal tissues. The term antibody shall include single-domain antibody, also known as nanobody.

The antibody may be polyclonal or monoclonal. Examples of monoclonal antibodies useful in the present invention is selected from the group consisting of, but not limited to, Rituximab, Trastuzumab, Cetuximab, LymphoCide, Vitaxin, Lym-1 and Bevacizumab.

In a preferred embodiment, the monoclonal antibodies are selected from the group consisting of Rituximab, Trastuzumab, Cetuximab, LymphoCide, Vitaxin, Lym-1, and Bevacizumab.

An “affibody” is defined as a small and stable antigen-binding molecule that can be engineered to bind specifically to a large number of target proteins. Affibody molecules according to the present invention include anti-ErbB2 affibody molecule and anti-Fibrinogen affibody molecule and other affibodies.

The peptides useful in the present invention act as a targeting moiety to enable the nano-sized particles to specifically bind to a target tissue of undesirable growth, wherein the peptides are selected from the group consisting of, but not limited to, RGD, somatostatin and analogs thereof, and cell-penetrating peptides or peptides allowing for cellular internalization.

In one embodiment, the peptides are selected from the group consisting of RGD, somatostatin and analogs thereof, and cell-penetrating peptides.

### Administration

The present invention provides for administration by any suitable route that allows for circulation of the nano-particles. It will be appreciated that the preferred route will depend on the general condition and age of the subject to be treated, the nature of the condition to be treated and the chosen formulation of nano-particles. Appropriate dosage forms for such administration may be prepared by conventional techniques.

Nano-particles according to the present invention may also be administered locally such as directly into the target tissue or into adjacent tissues of the target tissue. Such local administration may be intratumor administration.

The nano-particles according to the present invention may be administered parenterally, that is by intravenous, intramuscular, intraspinal, subcutaneous, intraarterial, intracardiac, intraosseous, intradermal, intracisternal, intrathecal, intracerebral, transdermal, transmucosal, inhalational, epidural, sublingual, intravitreal, intranasal, intrarectal, intravaginal or intraperitoneal administration. Further, the parental administration may according to the present invention be performed by infusion or injection.

In a preferred embodiment of the present invention, the nano-particles are administered by infusion or parenteral administration.

In yet another preferred embodiment of the present invention, the nano-sized particles are administered by intravenous, intraarterial, intrathecal, subcutaneous, intramuscular or intraperitoneal injection.

The nano-particles according to the present invention may also be administered enterally, by any suitable route that allows for circulation of the nano-particles of the present invention, such as the oral, rectal, nasal, pulmonary, buccal or sublingual administration.

Further the nano-particles according to the present invention may be administered to a mucosal membrane of the individual subject of treatment, e.g. in the nose, vagina, eye, mouth, genital tract, lungs, gastrointestinal tract, or rectum, preferably the mucosa of the nose, mouth or rectum.

According to the present invention, nano-particles may also be administered by inhalation that is by intranasal and oral inhalation administration. Appropriate dosage forms for such administration, such as an aerosol formulation or a metered dose inhaler, may be prepared by conventional techniques.

In one embodiment of the present invention, the nano-sized particles are administered topically.

The nanoparticles may be administered as a bolus or an infusion given over a specific period of time, such as 1 minute or more, 5 minutes or more, 10 minutes or more, or over about 1 hour.

The nano-particles according to the invention may be administered with at least one other active compound. The nano-particles and compounds may be administered simultaneously, either as separate formulations or combined in a unit dosage form, or administered sequentially.

In one embodiment of the present invention, the kit of parts comprising the nano-sized particles is for simultaneous, sequential or separate administration.

The administration of the nano-sized particles according to the invention may be adjusted according to the toxicity and degree of detectable contrast agent delivered to the cells of undesirable growth. Thus, in one embodiment of the present invention, the nano-sized particles are administered one or more times to the individual, such as 1 time, 2 times, 3 times, 4 times, or more, such as about 10 times, about 20 times, about 30 times, about 40 times, or about 50 times within the same treatment sequence.

The dosage of nano-particles to be administered to a specific subject can be determined by the physician in charge, based on parameters such as the weight or corresponding surface area of the subject to be treated, the age and condition of the subject, and the size and location of the target tissue to be imaged and irradiated. In one embodiment, at least 0.001%, such as more than 0.01%, 0.05%, 0.1%, 0.3%, 0.5%, 1%, 1.5%, 2%, 3%, 5%, or 10%, of the injected dose of nano-particles per gram or cm³ (mL) of tissue, reach the target tissue in a human. In one embodiment, the delivered dose to the diseased tissue is at least 0.01 mg/mL, such as at least 0.01 mg/mL, at least 0.1 mg/mL, at least 0.5 mg/mL, at least 1 mg/mL, at least 5 mg/mL, at least 10 mg/mL, or at least 50 mg/mL. In particularly preferred embodiments the delivered dose to the diseased tissue is between 0.1 mg/mL and 1 mg/mL or between 1 mg/mL and 10 mg/mL.

### Preparation and Synthesis

The present invention to provide methods for synthesis or preparation of nano-sized particles as described herein.

Detectable compounds may be transported inside the nano-sized particles by use of a seed crystal or a salt with low solubility, which allows for precipitation or aggregation of the detectable compound. Such crystals include crystals of transition metals, rare earth metals, alkali metals, alkali earth metals, other metals, as defined by the periodic table, for example crystals of gold (Au), bismuth (Bi), iron (Fe), Barium (Ba) and Calcium (Ca), Gadolinium (Gd) or any salt of the above mentioned metals which is insoluble or has a low solubility.

Reducing agents for facilitation of the precipitation or aggregation of the detectable compound may also be used for synthesis or preparation of nano-sized particles according to the present invention. Such reducing agents include ascorbic acid, sodium acrylate, glucose, fructose, glyceraldehyde, lactose, arabinose, maltose, citric acid and acetol.

In a preferred embodiment of the present invention, the nano-sized particle is prepared by use of sodium acrylate, ascorbic acid or citric acid as reducing agent.

In one preferred embodiment of the present invention, the method for preparation of nano-sized particles comprises one or more of the following steps:
a) Gold nanoparticles are coated with a cationic charged molecular species such as cysteamine
b) Lipids such as DSPC/DSPG/DSPE-PEG2000 in the ratio 70:25:5, are mixed in organic solution by a) first dissolving them in chloroform b) drying them using a stream of nitrogen c) overnight removal of trace residues of organic solvent using an oil pump, to obtain a thin film of lipids.
c) The lipid film is hydrated for 60 min in a buffer solution containing cationic gold nanoparticles from step a, such as cationic 50 nm gold particles.
d) The liposomes are extruded through 100 nm polycarbonate filters giving liposomes where the majority is in the size range from 60 to 120 nm as evaluated by cryo-transmission electron microscopy.
e) Empty liposomes are separated from gold nanoparticle liposomes by centrifugation

In another preferred embodiment of the present invention, the method for preparation of nano-sized particles comprises one or more of the following steps:
a) Gold nanoparticles are coated with a cationic charged molecular species such as cysteamine
b) The obtained cationic gold nanoparticles is added to a solution containing a negatively charged polymer of at least 10000 Daltons, such as hyaluronic acid and stirred for 1 hour.
   c) The particles are washed 3× by centrifugation by exchanging the buffer solution after each cycle.

In another embodiment of the present invention, one ore more ionophores are used for transportation of the contrast agent or a detectable compound inside the nano-sized particle. The term “ionophore” as used herein refers to any compound capable of forming a complex with a detectable compound, such as a metal and hereafter transporting this complex to the inside a nano-sized particle, such as for example across a bilayer of a liposome.

Ionophores according to the present invention may include 2-hydroxyquinoline (carbostyril), 8-hydroxyquinoline (oxine); 8-hydroxyquinoline β-D-galactopyranoside; 8-hydroxyquinoline β-D-glucopyranoside; 8-hydroxyquinoline glucuronide; 8-hydroxyquinoline-5-sulfonic acid; 8-hydroxyquinoline-β-D-glucuronide sodium salt; 8-quinolinol hemisulfate salt; 8-quinolinol N-oxide; 2-amino-8-quinolinol; 5,7-dibromo-8-hydroxyquinoline; 5,7-dichloro-8-hydroxyquinoline; 5,7-diiodo-8-hydroxyquinoline; 5,7-dimethyl-8-quinolinol; 5-amino-8-hydroxyquinoline dihydrochloride; 5-chloro-8-quinolinol; 5-nitro-8-hydroxyquinoline; 7-bromo-5-chloro-8-quinolinol; N-butyl-2,2′-imino-di(8-quinolinol); 8-hydroxyquinoline benzoate; 2-benzyl-8-hydroxyquinoline; 5-chloro-8-hydroxyquinoline hydrochloride; 2-methyl-8-quinolinol; 5-chloro-7-iodo-8-quinolinol; 8-hydroxy-5-nitroquinoline; 8-hydroxy-7-iodo-5-quinolinesulfonic acid; 5,7-dichloro-8-hydroxy-2-methylquinoline, other quinoline consisting chemical compounds and derivative thereof, and other ionophores.

In a preferred embodiment of the present invention, the ionophores are selected from the group comprising 8-Hydroxyquinoline (Oxine) and derivatives thereof, 2-hydroxyquinoline and derivatives thereof, A23187, hexamethylpropylene amine oxime (HMPAO) and derivatives thereof, diisopropyl iminodiacetic acid diisopropyl iminodiacetic acid (DISIDA) and derivatives thereof.

A method according to the present invention for preparation of liposomes comprising CT contrast agents which comprises a step wherein an ionophore is used and may include one or more of the following steps:
a) Mixing lipids for example by first dissolving them in chloroform followed by drying to obtain a thin film of lipids.
b) Hydrating the lipid film with a buffer solution comprising a chemical compound that is capable of either reducing a metal salt to a metal in oxidation state zero, or form an insoluble salt with a metal compound in an oxidation state higher than zero or a combination of the reduction and low solubility salt formation.
c) Obtaining liposomes with a preferred size of 20 to 150 nm.
d) Exchanging the exterior buffer giving a buffer where a metal salt has high solubility.
e) Adding a solution containing a metal salt with high solubility in water and an ionophore.
f) Stirring the solution to ensure efficient loading.

In another embodiment of the present invention the method for preparation of nano-sized particles is for preparation of liposomes comprising a CT contrast agent and an agent in solution that can be visualized by MR, SPECT or PET, and includes the use of an ionophore and comprising one or more of the following steps:
a) Mixing lipids for example by first dissolving them in chloroform followed by drying to obtain a thin film of lipids.
b) Hydrating the lipid film with a buffer solution comprising a chemical compound that is capable of either reducing a metal salt to a metal in oxidation state zero or form an insoluble salt with a metal compound in an oxidation state higher than zero or a combination of the reduction and using low solubility salt formation. Said buffer in this step furthermore comprises a chelating agent that strongly binds an agent visible by MR, SPECT or PET.
c) Obtaining liposomes with a preferred size of 20 to 150 nm.
d) Exchanging the exterior buffer by a suitable method to a buffer where the employed metal salt for CT imaging and the metal salt for MR, SPECT or PET have high solubility.
e) Adding a solution containing a metal salt for CT-imaging with high solubility in water, and a metal salt for MR, SPECT or PET and an ionophore to the liposomes in solution.
f) Stirring the solution for at least 30 min to ensure efficient loading.

In another embodiment of the present invention the method for preparation of nano-sized particles is for preparation of liposomes with CT contrast agent with use of an ionophore and an agent that is covalently bound to the liposome membrane that can be visualized by MR, SPECT or PET and comprising one or more of the following steps:
a) Mixing lipids for example by first dissolving them in chloroform or a mixture of chloroform and methanol or other organic solvent, followed by drying to obtain a thin film of lipids. One of the lipid components comprising an agent that can be visualized by MR, SPECT or PET either by a covalently attached agent or a chelating agent that can entrap the agent, wherein the agent can be present in this step or be introduced in a later step.
b) Hydrating the lipid film with a buffer solution comprising a chemical compound that will either reduce a metal salt to a metal in oxidation state zero or form an insoluble salt with a metal compound in an oxidation state higher than zero, or a combination of the reduction and using low solubility salt formation.
c) Obtaining liposomes with a preferred size of 20 to 150 nm.
d) Exchanging the exterior buffer.
e) Add a solution containing a metal salt with high solubility in water and an ionophore.
f) Stirring the solution for at least 30 min to ensure efficient loading.

The methods for preparation may further include a purification step such as size exclusion chromatography using sephadex G50.

According to the present invention, oxidation states higher than zero include monovalent cations, divalent cations, trivalent cations, tetravalent cations, pentavalent cations, hexavalent cations and heptavalent cations.

According to the present invention, the obtaining of liposomes with a preferred size may be done by evaluation of the size by cryo-transmission electron microscopy, and homogenization and/or extrusion using polycarbonate filters.

Exchanging the exterior buffer can according to the above mentioned methods be done by using suitable method for instance dialysis, column chromatography, or centrifugation.

Agents visible by MR, SPECT or PET and used in the methods for preparation are radioactive, paramagnetic or ferromagnetic compounds as defined herein, such as for example isotopes of Gadolinium, Indium, Technetium or Copper.

The chelating agents of the present invention or the methods of the present invention can be a chelating agent that forms a chelating complex with the MR, SPECT and PET agent. Examples of chelators include, but are not limited to, 1,4,7,10-tetraazacyclododecane-1,4,7,10-tetraacetic acid (DOTA) and derivative thereof; 1,4,8,11-tetraazacyclotetradecane (cyclam) and derivative thereof; 1,4,7,10-tetraazacyclododecane (cyclen) and derivative thereof; 1,4-ethano-1,4,8,11-tetraazacyclotetradecane (et-cyclam) and derivative thereof; 1,4,7,11-tetra-azacyclotetradecane (isocyclam) and derivative thereof; 1,4,7,10-tetraazacyclotridecane ([13]aneN₄) and derivative thereof; 1,4,7,10-tetraazacyclododecane-1,7-diacetic acid (DO2A) and derivative thereof; 1,4,7,10-tetraazacyclododecane-1,4,7-triacetic acid (DO3A) and derivative thereof; 1,4,7,10-tetraazacyclododecane-1,7-di(methanephosphonic acid) (DO2P) and derivative thereof; 1,4,7,10-tetraazacyclododecane-1,4,7-tri(methanephosphonic acid) (DO3P) and derivative thereof; 1,4,7,10-tetraazacyclododecane-1,4,7,10-tetra(methanephosphonic acid) (DOTP) and derivative thereof; ethylenediaminetetraacetic acid (EDTA) and derivative thereof; diethylenetriaminepentaacetic acid (DTPA) and derivative thereof; 1,4,8,11-tetraazacyclotetradecane-1,4,8,11-tetraacetic acid (TETA) and derivative thereof, or other adamanzanes and derivates thereof.

According to the present invention, the stirring of a solution comprising liposomes, metal salt and an ionophore can be done at least 30 min, such as at least 3 hours, such as at least 12 hours.

Further, according to the present invention, the stirring of a solution comprising liposomes, metal salt and an ionophore is done at a suitable temperature for efficient loading. Such a temperature includes at least 10° C. such as at least 20° C., such as at least 30° C., such as at least 40° C., such as at least 50° C., such as at least 60° C. and less than 95° C.

With the terms “loading”, “encapsulation”, or “entrapment” as used herein, is referred to an incorporation of detectable compounds into the interior of nano-sized particle compositions. With the terms “loading efficiency”, “entrapment efficiency” or “encapsulation efficiency” as used herein interchangeably, is referred to the fraction of incorporation of detectable compounds into the interior of nano-sized particle compositions expressed as a percentage of the total amount by weight of detectable compounds used in the preparation except water. With the term “encapsulation stability”, “storage stability” or “serum stability” is referred to a stability test of the nano-sized particle composition to measure the degree of leakage and/or release of the entrapped detectable compounds inside the nano-sized particle composition.

Determination of loading efficiency can be by weight or using MS methods such as ICP-MS, ICP-AES or AAS, or by spectroscopic methods such as UV or other methods known in the art.

In the methods for preparation according to the present invention, the loading efficiency measured in weight percent of the contrast agent compared to lipid is at least 50 wt/wt %, such as at least 60 wt/wt %, or such as at least 70 wt/wt %, or such as at least 80 wt/wt %, or such as at least 90 wt/wt %, or such as at least 95 wt/wt %, or such as at least 97 wt/wt %, or such as at least 98 wt/wt %, or such as at least 99 wt/wt %, or such as at least 99.9 wt/wt %.

According to the present invention, the metals used in preparation of nano-particles include transition metals, rare earth metals, alkali metals, alkali earth metals, other metals, as defined by the periodic table. The metals should be CT contrast agents in the employed form.

In a preferred embodiment of the present invention, the method for preparation of liposomes comprising gold particles comprises one or more of the following steps:
a) Lipids are mixed in organic solution, such as DSPC/Chol/DSPE-PEG2000 in the ratio 50:40:10 by first dissolving them in chloroform followed by drying using a stream of nitrogen followed by overnight removal of trace residues of organic solvent using an oil pump, to obtain a thin film of lipids.
b) The lipid film is hydrated for 60 min in a buffer solution containing sodium citrate and a small quantity of citrate stabilized gold nanoparticles with a diameter of 2-4 nm. These gold nanoparticles act as seed crystals inside the liposomes.
c) The liposomes are extruded through 100 nm polycarbonate filters giving liposomes where the majority is in the size range from 60 to 140 nm as evaluated by cryo-transmission electron microscopy.
d) The exterior buffer is exchanged with a buffer system that does not contain citrate by size exclusion chromatography using sephadex G50.
e) A buffer solution of HAuCl₄ is added to the liposome solution together with oxine.
f) The solution is stirred at least 3 hours at 50° C.
g) The liposomes are purified by size column chromatography using sephadex G50.

Hydroxyapatite occurs in bones and is a naturally occurring form of calcium apatite that is a well-functioning CT contrast agent. Calcium can be loaded into liposomes by the help of an ionophore.

In another preferred embodiment of the present invention, the method for preparation of nano-sized particles comprises one or more of the following steps:
d) Lipids such as DSPC/Chol/DSPE-PEG2000 in the ratio 50:40:10, are mixed in organic solution by a) first dissolving them in chloroform b) drying them using a stream of nitrogen c) overnight removal of trace residues of organic solvent using an oil pump, to obtain a thin film of lipids.
e) The lipid film is hydrated for 60 min in a buffer solution containing a high concentration of ammonium phosphate with pH adjusted to pH higher than 7, preferably 7.1, or 7.4, or 8.0, or 9.0.
f) The liposomes are extruded through 100 nm polycarbonate filters giving liposomes where the majority is in the size range from 60 to 140 nm as evaluated by cryo-transmission electron microscopy.
g) The exterior buffer is exchanged with a buffer system that does not contain ammonium phosphate by size exclusion chromatography using sephadex G50.
h) A buffer solution of calcium nitrate is added to the liposome solution together with oxine.
i) The solution is stirred at least 3 hours at 50° C.
j) The liposomes are purified by size column chromatography using sephadex G50

In a preferred embodiment of the present invention, the nano-sized particles produced as described above are administered to an individual as part of a method for treatment which comprises imaging and radiotherapy according to the present invention.

### EXAMPLES

### Example I

### Preparations of Liposomes According to the Present Invention

a. General Example of Preparation Method of Liposomes with Use of Ionophore

If the CT contrast agent is loaded into liposomes by the help of an ionophore the preferred preparation process comprises the steps of:
a) Mixing lipids of choice, e.g. by first dissolving them in chloroform followed by drying to obtain a thin film of lipids.
b) Hydrating the lipid film with a buffer solution that contains a chemical compound that will either reduce a metal salt to a metal in oxidation state zero or form an insoluble salt with a metal compound in an oxidation state higher than zero, e.g. +1, +2, +3, . . . , or a combination of the reduction and using low solubility salt formation.
c) Utilizing a method to obtain liposomes with a preferred size of 20 to 150 nm as evaluated by cryo-transmission electron microscopy, e.g. homogenization and/or extrusion.
d) Exchanging the exterior buffer by a suitable method, e.g. dialysis, column chromatography, or centrifugation giving a buffer where a metal salt has high solubility.
e) Adding a solution containing a metal salt with high solubility in water and an ionophore.
f) Stirring solution for at least 30 min, or at least 3 hours, or at least 12 hours, at a suitable temperature for efficient loading, e.g. 10, or 20, or 30, or 40, or 50, or more than 60 and less than 95° C.

A purification step can optionally be employed, e.g. size exclusion chromatography using sephadex G50.

Loading efficiency should be at least 50 wt/wt % of the contrast agent compared to lipid. Determination of loading efficiency can be by weight or using MS methods such as ICP-MS, ICP-AES or AAS, or by spectroscopic methods such as UV.

Metals include: Transition metals, rare earth metals, alkali metals, alkali earth metals, other metals, as defined by the periodic table. The metals should be CT contast agents in the employed form.

Ionophores include but are not limited to: 8-Hydroxyquinoline (Oxine) and derivatives thereof, 2-hydroxyquinoline and derivatives thereof, A23187, hexamethylpropylene amine oxime (HMPAO) and derivatives thereof, diisopropyl iminodiacetic acid diisopropyl iminodiacetic acid (DISIDA) and derivatives thereof.

b. Specific Example of Remote Loading of Gold Using Ionophore and Citrate as a Reducing Agent

By using the method below, Au(0) CT contrast agent is formed within liposomes by the help of a ionophore.

The process comprises the steps of:
a) Lipids are mixed in organic solution, e.g DSPC/Chol/DSPE-PEG2000 in the ratio 50:40:10 by first dissolving them in chloroform followed by drying using a stream of nitrogen followed by overnight removal of trace residues of organic solvent using an oil pump, to obtain a thin film of lipids.
b) The lipid film is hydrated for 60 min in a buffer solution containing sodium citrate and a small quantity of citrate stabilized gold nanoparticles with a diameter of 2-4 nm. These gold nanoparticles act as seed crystals inside the liposomes.
c) The liposomes are extruded through 100 nm polycarbonate filters giving liposomes where the majority is in the size range from 60 to 140 nm as evaluated by cryo-transmission electron microscopy.
d) The exterior buffer is exchanged with a buffer system that does not contain citrate by size exclusion chromatography using sephadex G50.
e) A buffer solution of HAuCl₄ is added to the liposome solution together with oxine.
f) The solution is stirred at least 3 hours at 50° C.
g) The liposomes are purified by size column chromatography using sephadex G50.
   c. Example of remote loading of Calcium using ionophore giving precipitation of low solubility hydroxyapatite

Hydroxyapatite occurs in bones and is a naturally occurring form of calcium apatite that is a well-functioning CT contrast agent. Calcium can be loaded into liposomes by the help of an ionophore.

The process may comprise the steps of:
a) Lipids are mixed in organic solution, e.g DSPC/Chol/DSPE-PEG2000 in the ratio 50:40:10 by first dissolving them in chloroform followed by drying using a stream of nitrogen followed by overnight removal of trace residues of organic solvent using an oil pump, to obtain a thin film of lipids.
b) The lipid film is hydrated for 60 min in a buffer solution containing a high concentration of ammonium phosphate with pH adjusted to pH higher than 7, preferably 7.1, or 7.4, or 8.0, or 9.0.
c) The liposomes are extruded through 100 nm polycarbonate filters giving liposomes where the majority is in the size range from 60 to 140 nm as evaluated by cryo-transmission electron microscopy.
d) The exterior buffer is exchanged with a buffer system that does not contain ammonium phosphate by size exclusion chromatography using sephadex G50.
e) A buffer solution of calcium nitrate is added to the liposome solution together with oxine.
f) The solution is stirred at least 3 hours at 50° C.
g) The liposomes are purified by size column chromatography using sephadex G50
   d. Example of Preparation Method of Liposomes with CT Contrast Agent and an Agent in Solution that can be Visualized by MR, SPECT or PET with Use of a Ionophore

CT contrast agent is loaded into liposomes by the help of an ionophore. The method comprises steps of:
a) Mixing lipids of choice, e.g. by first dissolving them in chloroform followed by drying to obtain a thin film of lipids.
b) Hydrating the lipid film with a buffer solution that contains a chemical compound that will either reduce a metal salt to a metal in oxidation state zero or form an insoluble salt with a metal compound in an oxidation state higher than zero, e.g. +1, +2, +3, . . . , or a combination of the reduction and using low solubility salt formation. The buffer solution furthermore contains a chelating agent that strongly binds an agent visible by MR, SPECT or PET, such as Gadolinium, Technetium such as technetium-99m, or Copper such as ⁶⁴Cu.
c) Utilize a method to obtain liposomes with a preferred size of 20 to 150 nm as evaluated by cryo-transmission electron microscopy, e.g. homogenization and/or extrusion.
d) Exchange the exterior buffer by a suitable method, e.g. dialysis, column chromatography, or centrifugation giving a buffer where the employed metal salt for CT imaging and the metal salt for MR, SPECT or PET have high solubility.
e) Add a solution containing a metal salt for CT with high solubility in water, and a metal salt for MR, SPECT or PET and an ionophore.
f) Stir solution for at least 30 min, or at least 3 hours, or at least 12 hours, at a suitable temperature for efficient loading, e.g. 10, or 20, or 30, or 40, or 50, or more than 60 and less than 95° C.
g) A purification step can optionally be employed, e.g. size exclusion chromatography using sephadex G50
h) Loading efficiency is measured to be at least 50 wt/wt % of the contrast agent compared to lipid. Determination of loading efficiency is done by weight or using MS methods such as ICP-MS, ICP-AES or AAS, or by spectroscopic methods such as UV.

Metals include: Transition metals, rare earth metals, alkali metals, alkali earth metals, other metals, as defined by the periodic table. The metals should be CT contrast agents in the employed form.

Ionophores include but are not limited to: 8-Hydroxyquinoline (Oxine) and derivatives thereof, 2-hydroxyquinoline and derivatives thereof, A23187, hexamethylpropylene amine oxime (HMPAO) and derivatives thereof, diisopropyl iminodiacetic acid diisopropyl iminodiacetic acid (DISIDA) and derivatives thereof.

The chelating agent component of is a chelating agent that forms a chelating complex with the MR, SPECT and PET agent. Examples of chelators include, but are not limited to, 1,4,7,10-tetraazacyclododecane-1,4,7,10-tetraacetic acid (DOTA) and derivative thereof; 1,4,8,11-tetraazacyclotetradecane (cyclam) and derivative thereof; 1,4,7,10-tetraazacyclododecane (cyclen) and derivative thereof; 1,4-ethano-1,4,8,11-tetraazacyclotetradecane (et-cyclam) and derivative thereof; 1,4,7,11-tetra-azacyclotetradecane (isocyclam) and derivative thereof; 1,4,7,10-tetraazacyclotridecane ([13]aneN₄) and derivative thereof; 1,4,7,10-tetraazacyclododecane-1,7-diacetic acid (DO2A) and derivative thereof; 1,4,7,10-tetraazacyclododecane-1,4,7-triacetic acid (DO3A) and derivative thereof; 1,4,7,10-tetraazacyclododecane-1,7-di(methanephosphonic acid) (DO2P) and derivative thereof; 1,4,7,10-tetraazacyclododecane-1,4,7-tri(methanephosphonic acid) (DO3P) and derivative thereof; 1,4,7,10-tetraazacyclododecane-1,4,7,10-tetra(methanephosphonic acid) (DOTP) and derivative thereof; ethylenediaminetetraacetic acid (EDTA) and derivative thereof; diethylenetriaminepentaacetic acid (DTPA) and derivative thereof; 1,4,8,11-tetraazacyclotetradecane-1,4,8,11-tetraacetic acid (TETA) and derivative thereof, or other adamanzanes and derivates thereof.

e. Example of Preparation Method of Liposomes with CT Contrast Agent with Use of a Ionophore and an Agent that is Covalently Bound to the Liposome Membrane that can be Visualized by MR, SPECT or PET

The CT contrast agent is loaded into liposomes by the help of an ionophore the process comprises the steps of:
a) Mixing lipids by first dissolving them in chloroform or a mixture of chloroform and methanol or other organic solvent, followed by drying to obtain a thin film of lipids. One of the lipid components comprise an agent that can be visualized by MR, SPECT or PET either by a covalently attached agent or a chelating agent that can entrap the agent. The agent can be present in this step or be introduced in a later step.
b) Hydrating the lipid film with a buffer solution that contains a chemical compound that will either reduce a metal salt to a metal in oxidation state zero or form an insoluble salt with a metal compound in an oxidation state higher than zero, e.g. +1, +2, +3, . . . , or a combination of the reduction and using low solubility salt formation.
c) Utilize a method to obtain liposomes with a preferred size of 20 to 150 nm as evaluated by cryo-transmission electron microscopy, e.g. homogenization and/or extrusion.
d) Exchange the exterior buffer by a suitable method, e.g. dialysis, column chromatography, or centrifugation giving a buffer where a metal salt has high solubility.
e) Add a solution containing a metal salt with high solubility in water and an ionophore.
f) Stir solution for at least 30 min, or at least 3 hours, or at least 12 hours, at a suitable temperature for efficient loading, e.g. 10, or 20, or 30, or 40, or 50, or more than 60 and less than 95° C.
g) A purification step can optionally be employed, e.g. size exclusion chromatography using sephadex G50
h) Loading efficiency should be at least 50 wt/wt % of the contrast agent compared to lipid. Determination of loading efficiency can be by weight or using MS methods such as ICP-MS, ICP-AES or AAS, or by spectroscopic methods such as UV.

Metals include transition metals, rare earth metals, alkali metals, alkali earth metals, other metals, as defined by the periodic table. The metals should be CT contast agents in the employed form.

Ionophores comprise 8-Hydroxyquinoline (Oxine) and derivatives thereof, 2-hydroxyquinoline and derivatives thereof, A23187, hexamethylpropylene amine oxime (HMPAO) and derivatives thereof, diisopropyl iminodiacetic acid diisopropyl iminodiacetic acid (DISIDA) and derivatives thereof.

The chelating agent can be a derivative with a functional handle suitable for covalently attachment to lipids of 1,4,7,10-tetraazacyclododecane-1,4,7,10-tetraacetic acid (DOTA) and derivative thereof; 1,4,8,11-tetraazacyclotetradecane (cyclam) and derivative thereof; 1,4,7,10-tetraazacyclododecane (cyclen) and derivative thereof; 1,4-ethano-1,4,8,11-tetraazacyclotetradecane (et-cyclam) and derivative thereof; 1,4,7,11-tetra-azacyclotetradecane (isocyclam) and derivative thereof; 1,4,7,10-tetraazacyclotridecane ([13]aneN₄) and derivative thereof; 1,4,7,10-tetraazacyclododecane-1,7-diacetic acid (DO2A) and derivative thereof; 1,4,7,10-tetraazacyclododecane-1,4,7-triacetic acid (DO3A) and derivative thereof; 1,4,7,10-tetraazacyclododecane-1,7-di(methanephosphonic acid) (DO2P) and derivative thereof; 1,4,7,10-tetraazacyclododecane-1,4,7-tri(methanephosphonic acid) (DO3P) and derivative thereof; 1,4,7,10-tetraazacyclododecane-1,4,7,10-tetra(methanephosphonic acid) (DOTP) and derivative thereof; ethylenediaminetetraacetic acid (EDTA) and derivative thereof; diethylenetriaminepentaacetic acid (DTPA) and derivative thereof; 1,4,8,11-tetraazacyclotetradecane-1,4,8,11-tetraacetic acid (TETA) and derivative thereof, or other adamanzanes and derivates thereof.

### Example II

### Preparation of Nano-Sized Particles Useful in the Methods of the Present Invention

a. Procedure for Obtaining Gold Nanoparticle (AuNP) Synthesis of Different Sizes from 16-80 nm

### Materials:

Hydrogen Tetrachloroaurate(III) Tetrahydrate was purchased from Wako Pure Chemical Industries Ldt. Sodium acrylate, sodium hydroxide, nitric acid and hydrochloric acid was purchased from Sigma-Aldrich. MilliQ water was used throughout the preparation of gold nanoparticles (Millipore, Bedford, Mass.). All materials were used without further purification.

### Characterization:

The particles was characterized by dynamic light scattering and zeta potential measurements (Zetasizer Nano; Malvern Instruments, Malvern, UK) as well as by their UV-vis spectra (Unicam Helios Uni-9423). A Tecnai T20 G2 (FEI Company, USA) transmission electron microscope and an atomic force microscope (PSIA XE 150 Park Systems, Korea) were used to visualize the size and homogeneity of the particles.

### Synthesis:

### 16 nm AuNP

Glassware and magnet were washed in aqua regia (HCl:HNO₃3:1) and rinsed extensively with MilliQ water. HAuCl₄×3H₂O (156.8 mg) was dissolved in MilliQ water (380.8 mL), fitted with a condenser and heated to reflux in an oil bath. A preheated (˜70° C.) solution of sodium acrylate (859 mg, 80 mM, 114.2 mL) was added and the reaction was allowed to reflux for one hour. The reaction undergoes a color change from clear to purple and finally wine red. The reaction was cooled to room temperature.

DLS: 27.6 nm, PDI: 0.096; Zeta: −25.85 mV±1.43 mV; UV-vis: λₘₐₓ 526 nm; TEM 16-20 nm; AFM 16-20 nm.

### 30 nm AuNP

Glassware was washed in aqua regia (HCl:HNO₃ 3:1) and rinsed extensively with MilliQ water.

HAuCl₄×3H₂O (125.2 mg) was dissolved in MilliQ water (1.34 L) and the pH adjusted to 7 using a 0.1 M sodium hydroxide solution. Sodium acrylate (1.72 g, 446.7 mL, 41 mM) in MilliQ water was added to the pH adjusted solution, the flask swirled shortly and left at room temperature for 3-4 days. The wine red color developed slowly during these days. The reaction was monitored by the intensity (OD) in the UV-vis spectra. The concentration of the AuNPs was increased to ˜0.8 mM by centrifugation (6500 rpm, 10 minutes).

DLS: 32.8 nm, PDI: 0.050; Zeta: −32.94 mV±1.0 mV; UV-vis: λₘₐₓ 523 nm; TEM 30 nm; AFM 30 nm.

### 50 nm AuNP

AuNP at a size of 30 nm was used as seeds to grow 50 nm AuNP. Glassware was washed in aqua regia (HCl:HNO₃ 3:1) and rinsed extensively with MilliQ water. HAuCl₄×3H₂O (64 mg) was dissolved in MilliQ water (546 mL) and the pH adjusted to 7 using a 0.1 M sodium hydroxide solution. Seeds of 30 nm were added in the concentration of 1.17×10¹¹ nanoparticles/mL followed by a solution of sodium acrylate (876.3 mg, 182 mL, 51.2 mM). Volumetric ratios used was (Au³⁺:Au⁰:Sodium acrylate): (6:2:2). The flask was swirled shortly and left at room temperature for 3-4 days. Reaction was monitored by growth of the particles by DLS. The concentration of the AuNPs was increased to ˜0.8 mM by centrifugation (6500 rpm, 10 minutes). DLS: 52.6 nm, PDI: 0.126; Zeta: −40.21 mV±1.62 mV; UV-vis: λₘₐₓ 531 nm; TEM 50 nm; AFM 50 nm.

### 80 nm AuNP

AuNP at a size of 50 nm was used as seeds to grow 80 nm AuNP. Same procedure as for the growth of 50 nm AuNP was used. The particles were concentrated by centrifugation at 4300 rpm for 10 minutes.

DLS: 85.4 nm, PDI: 0.047; Zeta: −50.31 mV±1.58 mV; UV-vis: λₘₐₓ 557 nm; TEM 80 nm; AFM 80-85 nm.

b. PEG Polymer Coated Gold Nanoparticle for CT Imaging

Gold nanoparticles are synthesized with a PEG coating by further reaction with the solutions obtained in example IIa. Thiol functionalized monomethoxy poly(ethylene glycol) in the size range of PEG₂₀₀₀ to PEG₁₀₀₀₀ were purchased from Rapp Polymere. The PEGylated gold nanoparticles are collected by centrifugation and washed with MQ water or buffer.

PEGylation procedure 16 nm AuNP: Excess of mPEG thiol (8 PEG molecules pr. nm² surface) was added to a 16 nm AuNP solution and the reaction was left at room temperature to stir over night. The AuNP was collected by centrifugation at 9500 rpm for 40 minutes

Pegylation procedure 30 nm AuNP: mPEG thiol (8 PEG molecules pr. nm² surface) was added to a solution of 30 nm AuNP and was allowed to stir over night before collecting the AuNPs by centrifugation at 9500 rpm for 20 minutes.

Pegylation procedure 50 nm AuNP: mPEG thiol (8 PEG molecules pr. nm² surface) was added to a solution of 50 nm AuNP and was allowed to stir over night. The AuNP was collected by centrifugation at 9500 rpm for 10 minutes.

Pegylation procedure 80 nm AuNP: mPEG thiol (8 PEG molecules pr. nm² surface) was added to the AuNP and the mixture was allowed to stir over night The particles were collected by centrifugation at 9000 rpm for 10 minutes.

c. Pegylated Gold Nanorods

Highly stable 13×47 nm cetyltrimethylammonium bromide (CTAB)-coated gold nanorods (from Nanopartz) are centrifuged at 16,000 rcf to concentrate the rods where after they are resuspended in a solution of MeO-PEG-SH (5 kDa) The nanorods can be collected by centrifugation after which they are washed successively with MQ water.

d. Polymer-Coated Bismuth Sulphide Nanoparticles

Bismuth sulphide nanocrystals is prepared by precipitation in the presence of a surfactant. A bismuth-thiolate solution is prepared by adding 3-mercaptopropionic acid to bismuth citrate in NH₄OH. Sodium sulphide is added dropwise to the bismuth-thiolate solution under vigorous stirring. The mixture is filtered and the product lyophilized. The product is dissolved in aqueous polyvinylpyrrolidone (PVP) and dialysed against aqueous polyethyleneoxide resulting in PVP-coated nanoparticles.

e. A Calcium Phosphate Liposome Core-Shell Nanocomposite

Preparation of a liposome core-shell nanocomposites is achieved by dissolving soybean lecithin in chloroform is dried to form a lipid thin film. A Ca(NO₃)₂.4H₂O and (NH₄)₂HPO₄ solution adjusted to pH 2.4 with HNO₃ is then used to hydrate the dry lipid film to form liposomes. The vesicle suspension is emulsified by emulsiflex-B3 (Avestin, Canada) ten times. To obtain liposomes of uniform size, the solution is then extruded through polycarbonate membrane filters (Poretics, USA) with a pore diameter of 200 nm. The extrusion is repeated 10 times. The suspension is passed through an Na⁺ ion exchange column to remove unencapsulated Ca²⁺. The pH is adjusted to 10 with NH₄OH solution which drives the precipitation process within the liposomes due to slow diffusion of hydroxide to the liposome interior.

f. Dendrimers of PAMAM with Entrapped Gold Nanoparticles for CT Imaging

HAuCl₄ is added to PAMAM dendrimer containing a seed gold nanoparticle, e.g. 2 nm particle, after which ascorbic acid is added at once and reacted for 30 min. The mild reduction by ascorbic acid secures growth of the gold seed to a larger gold nanoparticle within the dendrimer that can be used in CT imaging.

g. Nanoparticles are PEG Polymer Coated Gold Nanoparticles for CT Imaging Combined with MR or PET Imaging

According to the two following examples the chelating agent is a derivative with a linker containing a thiol group of 1,4,7,10-tetraazacyclododecane-1,4,7,10-tetraacetic acid (DOTA) and derivative thereof; 1,4,8,11-tetraazacyclotetradecane (cyclam) and derivative thereof; 1,4,7,10-tetraazacyclododecane (cyclen) and derivative thereof; 1,4-ethano-1,4,8,11-tetraazacyclotetradecane (et-cyclam) and derivative thereof; 1,4,7,11-tetra-azacyclotetradecane (isocyclam) and derivative thereof; 1,4,7,10-tetraazacyclotridecane ([13]aneN₄) and derivative thereof; 1,4,7,10-tetraazacyclododecane-1,7-diacetic acid (DO2A) and derivative thereof; 1,4,7,10-tetraazacyclododecane-1,4,7-triacetic acid (DO3A) and derivative thereof; 1,4,7,10-tetraazacyclododecane-1,7-di(methanephosphonic acid) (DO2P) and derivative thereof; 1,4,7,10-tetraazacyclododecane-1,4,7-tri(methanephosphonic acid) (DO3P) and derivative thereof; 1,4,7,10-tetraazacyclododecane-1,4,7,10-tetra(methanephosphonic acid) (DOTP) and derivative thereof; ethylenediaminetetraacetic acid (EDTA) and derivative thereof; diethylenetriaminepentaacetic acid (DTPA) and derivative thereof; 1,4,8,11-tetraazacyclotetradecane-1,4,8,11-tetraacetic acid (TETA) and derivative thereof, or other adamanzanes and derivates thereof.

h. PEG Polymer Coated Gold Nanoparticle for CT Imaging and MR Imaging

Gold nanoparticles are synthesized with a PEG coating by heating a solution of HAuCl₄ for 10 min before rapid addition of sodium citrate to the solution under vigorous stirring. After cooling the solution an appropriate length MeO-PEG-SH is added, e.g. PEG2000-SH, together with a thiol derivatized chelating agent that will bind a metal that can be visualized using MR imaging. This mixture is stirred for 1 hour. The MR imaging agent is added, e.g. Gadolinium and the solution is stirred 1 hour. The PEGylated gold nanoparticles are collected by centrifugation and washed with MQ water.

i. PEG Polymer Coated Gold Nanoparticle for CT Imaging and PET Imaging

Gold nanoparticles are synthesized with a PEG coating by heating a solution of HAuCl₄ for 10 min before rapid addition of sodium citrate to the solution under vigorous stirring. After cooling the solution an appropriate length MeO-PEG-SH is added, e.g. PEG2000-SH, together with a thiol-derivatized chelating agent that will bind a metal that can be visualized using PET imaging. This mixture is stirred for 1 hour. The PEGylated gold nanoparticles are collected by centrifugation and washed with MQ water. The PET imaging agent is added, e.g. Copper (⁶⁴Cu) e.g. in PBS buffer, and the solution is stirred 30 min.

### Example III

### Preparation of Lipid-Coated Nano-Sized Particles Useful in the Methods of the Present Invention

This Example describes the synthesis of a lipid-coated nano-sized particle.

### Step 1: Synthesis of 50 Nm Gold Nano-Sized Particle (AuNP)

Glassware was washed in aqua regia (HCl:HNO₃ 3:1) and rinsed extensively with MilliQ water.

HAuCl₄×3H₂O (125.2 mg) was dissolved in MilliQ water (1.34 L) and the pH adjusted to 7 using a 0.1 M sodium hydroxide solution. Sodium acrylate (1.72 g, 446.7 mL, 41 mM) in MilliQ water was added to the pH adjusted solution, the flask swirled shortly and left at room temperature for 3-4 days. The wine red color developed slowly during these days. The reaction was monitored by the intensity (OD) in the UV-vis spectra. The AuNPs was concentrated by centrifugation at 6500 rpm for 10 minutes.

The obtained AuNP at a size of 30 nm was used as seeds to grow 50 nm AuNP. Glassware was washed in aqua regia (HCl:HNO₃ 3:1) and rinsed extensively with MilliQ water. HAuCl₄×3H₂O (64 mg) was dissolved in MilliQ water (546 mL) and the pH adjusted to 7 using a 0.1 M sodium hydroxide solution. Seeds of 30 nm were added in the concentration of 1.17×10¹¹ nanoparticles/mL followed by a solution of sodium acrylate (876.3 mg, 182 mL, 51.2 mM) and in the presence of 2-aminoethanethiol (HAuCl₄:2-aminoethanethiol ratio was 1:1.3). Volumetric ratios used was (Au³⁺:Au⁰:Sodium acrylate): (6:2:2). The flask was swirled shortly and left at room temperature for 3-4 days. Reaction was monitored by growth of the particles by DLS. The AuNP were collected and washed by centrifugation at 7500 rpm for 10 minutes.

The obtained cationic particle suspension was added to a lipid film of DSPC/DSPG/DSPE-PEG2000 (70:25:5) which was hydrated for 60 min at 70° C. The lipid gold particles were collected by centrifugation at 8500 rpm for 10 minutes and washed 3 times using this procedure by exchanging the supernatant.

### REFERENCES

1. Dawson L A, Sharpe M B. Image-guided radiotherapy: rationale, benefits, and limitations. Lancet Oncol. 2006 October; 7(10):848-58.
2. Jaffray, D A; J P Bissonnette, T Craig (1999) [1999]. “X-ray Imaging for Verification and Localization in Radiation Therapy in Modern Technology of Radiation Oncology (suppl. 1)”. Modern Technology of Radiation Oncology. Madison, Wis.: Medical Physics Pub
3. Hainfeld J F, Slatkin D N, Focella T M, Smilowitz H M. Gold nanoparticles: a new X-ray contrast agent. Br J Radiol. 2006 March; 79(939):248-53.
4. Kim D, Park S, Lee J H, Jeong Y Y, Jon S. Antibiofouling polymer-coated gold nanoparticles as a contrast agent for in vivo X-ray computed tomography imaging. J Am Chem Soc. 2007 Jun. 20; 129(24):7661-5.
5. Rabin O, Manuel Perez J, Grimm J, Wojtkiewicz G, Weissleder R. An X-ray computed tomography imaging agent based on long-circulating bismuth sulphide nanoparticles. Nat Mater. 2006 February; 5(2):118-22.
6. Chu M and Liu G. Preparation and characterization of hydroxoapatite/liposome core-shell nanocomposites. Nanotechnology 16, 2005, 1208-1212.
7. Haba Y, Kojima C, Harada A, Ura T, Horinaka H, Kono K. Preparation of poly(ethylene glycol)-modified poly(amido amine) dendrimers encapsulating gold nanoparticles and their heat-generating ability. Langmuir. 2007 May 8; 23(10):5243-6.
8. Zheng J, Perkins G, Kirilova Am Allen C and Jaffray D A. Multimodal Contrast Agent for Combined Computed Tomography and Magnetic Resonance Imaging Applications. Investigative Radiology 41, 2006, 339-348.
9. Chithrani, D B; Dunne, M; Stewart, J., Allen, C; and Jaffray, D A. Cellular uptake and transport of gold nanoparticles incorporated in a liposomal carrier. Nanomedicine 2010; 6:161-169.

1. A composition comprising nano-sized particles comprising a solid form of a compound detectable by X-ray imaging for use in image-guided radiotherapy of a target tissue in an individual, the target tissue comprising undesirably growing cells.

2. The composition according to claim 1, wherein the image-guided radiotherapy comprises:
a) administering said composition to said individual;
b) recording X-ray images of the target tissue to obtain a definition of the target tissue; and
c) using the definition of the target tissue obtained in b) to direct radiotherapy to the target tissue;
wherein b) and c) are performed sequentially or simultaneously.

3. The composition according to claim 1, wherein the nano-sized particles have a half-life in circulation of at least 1 hour.

4. The composition according to claim 1, wherein the nano-sized particles have a number average diameter of 10 to 150 nm.

5. The composition according to claim 1, wherein the nano-sized particles are selected from the group consisting of liposomes, polymersomes, dendrimers, water-soluble cross-linked polymers, hydrocolloids, micelles, coated metal particles, and coated particles wherein the core is a solid salt.

6. The composition according to claim 1, wherein the nano-sized particles are liposomes.

7. The composition according to claim 1, wherein the nano-sized particles are coated particles where the core comprises a solid metal and/or a solid metal salt.

8. The composition according to claim 1, wherein the nano-sized particles comprise a shell or surface coat comprising polyethylene glycol (PEG).

9. The composition according to claim 1, wherein the detectable compound is at least 10 weight percent of the nano-sized particle, excluding any water.

10. The composition according to claim 1, wherein the detectable compound is in the form of a solid metal or a solid metal salt and comprises one or more isotopes selected from the group consisting of gold (Au), bismuth (Bi), iron (Fe), Barium (Ba), Calcium (Ca), and Magnesium (Mg).

11. The composition according to claim 1, wherein the detectable compound is gold (Au) or bismuth (Bi), such as gold (Au).

12. The composition according to claim 1, wherein the target tissue comprises tumour cells.

13. The composition according to claim 2, wherein the administration of said composition in step a) allows for the recording of X-ray images in step b) for at least 3 days after step a), optionally wherein the nano-sized particles have a half-life in circulation of at least 8 hours.

14. The composition according to claim 1, wherein step b) in claim 2 results in a three or multi-dimensional coordinate data set, wherein the fourth dimension is time, said data set being used for the definition and treatment guidance of the target tissue.

15. The composition according to claim 1, wherein the X-ray imaging is computed tomography (CT) imaging.

16. The composition according to claim 1, wherein the nano-sized particle comprises a radioactive or paramagnetic compound for one or more imaging modalities such as magnetic resonance imaging (MRI), positron emission tomography (PET) imaging, single photon emission computed tomography (SPECT) imaging or nuclear scintigraphy imaging.

17. The composition according to claim 15, wherein the image-guided radiotherapy further comprises an imaging step with one or more imaging modalities selected from the group consisting of magnetic resonance imaging (MRI), positron emission tomography (PET) imaging, single photon emission computed tomography (SPECT) imaging, nuclear scintigraphy imaging, ultrasonography imaging, ultrasonic imaging, near-infrared imaging or fluorescence imaging.

18. A nano-sized particle for use in X-ray image recording, said particle comprising:
(i) a shell or surface coat comprising a lipid layer such as a lipid monolayer and/or a lipid bilayer; and
(ii) a core comprising a contrast agent for computed tomography (CT)-imaging, selected from the group consisting of gold (Au) and bismuth (Bi), wherein the contrast agent is in a solid form.

19. The composition according to claim 1, wherein the nano-sized particle is as defined in claim 18.

20. A method for treatment of a condition or disease associated with undesirable growth of cells in an individual in need thereof, wherein said method comprises the steps of:
a) providing nano-sized particles comprising a compound detectable by computed tomography (CT)-imaging,
b) administering the nano-sized particles to said individual,
c) recording computed tomography (CT)-images of a target tissue comprising the undesirably growing cells thereby obtaining a definition of the target tissue giving the precise location of the undesirably growing cells and separation from normal tissue,
d) using the definition of the target tissue obtained in c) to direct radiotherapy to the undesirably growing cells and save normal tissue,
wherein said compound is in solid form, and
wherein image-recording and execution of radio therapeutic treatment is integrated and performed sequentially or simultaneously.
